(19) United States
(12) Patent Application Publication
(10) Pub. No.: US 2019/0385752 A1
(43) Pub. Date: Dec. 19, 2019

(54) AUTOMATED PROVISIONING OF CLINICAL ADVICE ASSOCIATED WITH A PATIENT WITH DIABETES (PWD) AND RELATED SYSTEMS, METHODS, AND DEVICES

(71) Applicant: Bigfoot Biomedical, Inc., Milpitas, CA (US)

(72) Inventors: Jacob Bowland, Milpitas, CA (US); Bethany Leigh Salmon, San Jose, CA (US); Jennifer Martin Block, Menlo Park, CA (US); Alex Constantin, Milpitas, CA (US); Sabine Kabel-Eckes, Mountain View, CA (US); Jeff Boissier, San Jose, CA (US)

(21) Appl. No.: 16/442,238

(22) Filed: Jun. 14, 2019

Related U.S. Application Data
(60) Provisional application No. 62/790,405, filed on Jan. 9, 2019.
(60) Provisional application No. 62/686,556, filed on Jun. 18, 2018.
(60) Provisional application No. 62/786,215, filed on Dec. 28, 2018.

(51) Int. Cl. G16H 80/00; G16H 50/20; A61B 5/145; G16H 10/60; G16H 20/10; G16H 40/67; A61B 5/00

(52) U.S. Cl. CPC G16H 80/00; G16H 50/20; A61B 5/14532; G16H 40/63; G16H 20/10; G16H 40/67; A61B 5/7275; G16H 10/60

(57) ABSTRACT

A computer-implemented method for automated provisioning of clinical advice to a provider caring for a patient with diabetes (PWD). The method includes detecting a clinically relevant pattern in therapy data of a patient under the care of a provider, and identifying a predefined behavior of a PWD responsive to the detected clinically relevant pattern. The clinically relevant detected pattern is indicative of desirable behaviors and/or undesirable behaviors. The method further includes selecting a therapy insight associated with the identified predefined behavior, wherein the therapy insight comprises clinical advice associated with the PWD that includes a behavior recommendation, and responsive to the selecting the therapy insight, automatically sending, to a provider-dashboard associated with the provider caring for the PWD, the selected therapy insight.

### CROSS-REFERENCE TO RELATED APPLICATIONS

This application claims the benefit of the filing date of U.S. Provisional Patent Application Ser. No. 62/790,405, filed Jan. 9, 2019, for “Clinical Decision Support System, and Related Systems, Methods, and Devices,” the contents and disclosure of which is hereby incorporated herein in its entirety by this reference. This application also claims the benefit of the filing date of U.S. Provisional Patent Application Ser. No. 62/686,556, filed Jun. 18, 2018, for “Remote Monitoring Tools for Diabetes Management Systems, and Related Methods, Systems and Devices,” the contents and disclosure of which is hereby incorporated herein in its entirety by this reference.

### TECHNICAL FIELD

This disclosure relates to automated provisioning of clinical advice systems, methods, and devices adapted to collect and/or transmit data relating to clinical advice associated with a person with diabetes (PWD) and/or other therapy related data and to provide a user with therapy recommendations. In particular embodiments, automated provisioning of clinical advice systems, devices, and methods are disclosed, which may be utilized with insulin injection devices, including components adapted to provide a user with therapy insights based, at least in part on, identified predefined behaviors.

### BACKGROUND

Diabetes mellitus is a chronic metabolic disorder caused by the inability of a person's pancreas to produce sufficient amounts of the hormone insulin such that the person's metabolism is unable to provide for the proper absorption of sugar and starch. The inability to absorb those carbohydrates sometimes leads to hyperglycemia, i.e., the presence of an excessive amount of glucose within the blood plasma. Hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities.

Because healing is not yet possible, a permanent therapy is necessary which maintains a proper blood glucose level within normal limits. Maintaining a proper glucose level is achieved by regularly supplying insulin to a person with diabetes (PWD). Maintaining a proper blood glucose level creates a significant cognitive burden for a PWD and affects many aspects of the PWD's life. For example, the cognitive burden on a PWD can be attributed to, among other things, tracking meals and constant check-ins and minor course corrections of blood glucose levels. The adjustments of blood glucose levels by a PWD can include taking insulin, tracking insulin dosing and glucose, deciding how much insulin to take, how often to take it and how to time insulin doses in relation to meals and/or glucose fluctuations. These factors make up just a portion of the significant cognitive burden of a PWD.

The following example of a typical daily routine for a PWD further illustrates the significant cognitive burden of a PWD. In the morning, the first thoughts/actions by a PWD is often related to their glucose, such as, what is their blood glucose level? How was their blood glucose level overnight? And how are they currently feeling? Upon checking their blood glucose levels (e.g., using a blood glucose meter or monitor), a PWD can then consider what actions to take, such as adjusting their morning activities, changing when or what to eat for breakfast, or determining to take rapid-acting (RA) insulin. Before they even eat breakfast (or any meal), a PWD considers the amount of food and types of food they plan to eat, perhaps modifying their RA insulin dose based on the carbohydrate content of the food they choose to eat. Before they administer RA insulin, the PWD will try to remember when they took their last dose of insulin, what happened the last time they ate a particular meal and how they felt.

Before leaving the house, a PWD considers, among other things, whether they have enough supplies for glucose monitoring or insulin dosing. This can include batteries, charged devices, backup supplies, glucose testing supplies, and insulin supplies to treat for high blood glucose levels. Additionally, a PWD needs to consider any physical activities (e.g., walking kids to school, going to the gym, riding a bike) that will affect their glucose because exercise may cause their blood glucose to go lower than expected. Even before driving a vehicle, a PWD checks their glucose to determine if it is at a safe level for driving.

As lunchtime approaches, a PWD considers their glucose prior to eating lunch, such as what time they can expect to eat, what they expect to eat throughout the day. As such, a PWD tallies up the carbohydrates and adjusts insulin doses in their head. A PWD also considers what insulin doses were recently taken and whether those doses may still be working to lower blood glucose. This is all done in parallel with whatever they are doing in their busy day, and so the PWD often forgets or fails to fully consider all of the factors described above.

Throughout the day, a PWD often checks glucose levels, especially on days when their activities vary from a typical day. This constant thinking, checking, planning can be exhausting, especially when each check requires decisions, math, and possible behavior changes. Additionally, during the day, a PWD may check inventory on supplies, speak with a health care provider (HCP), refill prescriptions, contact their health insurance to discuss their therapy and/or supplies.

In the evening, after an exhausting day, a PWD may have to take a daily insulin dose of long-acting (LA) insulin. Additionally, the PWD may determine if their glucose is holding steady before they fall asleep. If they use an infusion pump, they have to check if their insulin pump is low on insulin and whether they need to refill it before sleep. If they have a continuous glucose monitor, they have to check and see if it is working. Even then, based on what they ate for dinner, the nighttime insulin might not keep their glucose steady. Glucose levels in the night can interfere with sleep as well as add anxiety that could disrupt sleep.

Accordingly, managing diabetes requires significant attention to detail throughout the day. Even with careful planning and self-monitoring, a PWD may skip doses, double dose, or dose the wrong amount and/or type of insulin. Insufficient insulin can result in hyperglycemia, and too much insulin can result in hypoglycemia, which can result in clumsiness, trouble talking, confusion, loss of consciousness, seizures, or death.

In order to assist with self-treatment, some diabetes treatment devices (e.g., blood glucose meters, insulin pumps, etc.) are equipped with insulin bolus calculators that have the user input an estimate (e.g., numerical estimate) of the quantity of carbohydrates consumed or about to be consumed (or additionally or alternatively protein, fat, or other meal data) and the bolus calculator outputs a recommended size for the insulin bolus dosage. Although bolus calculators remove some of the mental calculations that need to be made by the user in determining an appropriate insulin bolus dosage, bolus calculators still burden the user with the mental task of evaluating the constituents of their meal, may require the use of a secondary device, and often require manual entry of data.

A healthcare provider (HCP) (e.g., physician, endocrinologist) may assist the PWD in the self-treatment. For example, an HCP may assist the PWD via a dosing system used to connect PWDs with HCPs to improve awareness and knowledge with the goal to ultimately improve insulin therapy outcomes. Some conventional dosing systems provide recommendations to PWDs and HCPs for updating and changing insulin delivery settings and/or track blood glucose patterns, carbohydrate intake, and exercise, and provide summary information of the same. Some conventional dosing systems focus on type 2 diabetes with an emphasis on only basal rate, requesting food logging, requesting exercise logging, and information about past blood glucose highs and lows. However, some conventional dosing systems do not provide recommendations for improving or updating PWD behavior or therapy settings. Moreover, none focus on the ability to set an initial therapy recommendation and allow updating of the therapy recommendation. Additionally, some conventional dosing systems do not provide options for changing incorrect behaviors, nor do they provide recommendations for continuing correct behaviors; link patterns of behavior to specific types of dosing, more specifically, identify a behavior pattern and link it to long acting insulin doses or rapid acting insulin doses; or track whether a dose was given for multiple daily injections (MDI) or if the wrong dose was given.

Although conventional dosing systems may remove some of the mental burdens for the HCP and/or PWD in determining an appropriate recommendation related to insulin dosing, dosing systems still burden the HCP and/or PWD with the mental task of at least manually evaluating therapy data, manually determining a dosing recommendation, and often require manual entry of data.

### BRIEF DESCRIPTION OF THE DRAWINGS

The present disclosure may be understood more fully by reference to the following detailed description of example embodiments, which are illustrated in the accompanying figures.

FIG. 1A illustrates a clinical decision support (CDS) system according to embodiments of the present disclosure.

FIG. 1B illustrates a CDS system according to embodiments of the present disclosure.

FIG. 2A illustrates a list of patients presented to an HCP on HCP dashboard according to embodiments of the present disclosure.

FIG. 2B illustrates an overview of an insight presented to an HCP on HCP dashboard according to embodiments of the present disclosure.

FIG. 2C illustrates a glucose data view of an insight according to embodiments of the present disclosure.

FIG. 2D illustrates a statistics and insights view of an insight according to embodiments of the present disclosure.

FIG. 2E illustrates a settings view of an insight according to embodiments of the present disclosure.

FIG. 2F illustrates a recommendation history view of an insight according to embodiments of the present disclosure.

FIG. 3 illustrates workflow for selecting and sending recommendations to a PWD via HCP dashboard according to embodiments of the present disclosure.

FIG. 4 illustrates a view of various information associated with recommendations, therapy changes and/or care notes using an HCP dashboard according to embodiments of the present disclosure.

FIG. 5 illustrates an insight view displayed on an HCP dashboard according to an embodiment of the present disclosure.

FIG. 6 illustrates an insight view displayed on an HCP dashboard according to an embodiment of the present disclosure.

FIG. 7A illustrates a summary view presented at a PWD dashboard according to embodiments of the present disclosure.

FIG. 7B illustrates a view presented at a PWD dashboard according to embodiments of the present disclosure.

FIG. 7C illustrates a summary view of recommended therapy setting updates presented at a PWD dashboard according to embodiments of the present disclosure.

FIG. 7D illustrates a view of training modules presented at a PWD dashboard according to embodiments of the present disclosure.

FIG. 8 illustrates a method for automated provisioning of clinical advice to a provider caring for a PWD according to embodiments of the present disclosure.

FIG. 9 illustrates a method of accepting a recommendation by a PWD according to embodiments of the present disclosure.

FIG. 10 illustrates a therapy management system according to embodiments of the present disclosure.

FIG. 11A illustrates a therapy management system according to embodiments of the present disclosure.

FIG. 11B illustrates a therapy management system according to embodiments of the present disclosure.

FIG. 12 illustrates a method for managing therapy settings for a PWD according to embodiments of the present disclosure.

FIG. 13 illustrates a method for managing therapy settings for a PWD according to embodiments of the present disclosure.

### DETAILED DESCRIPTION

Improved systems for communication between HCPs and PWDs would reduce some of the cognitive burden on the PWD to manage their diabetes. A limiting factor is that HCPs and PWDs do not have a system that lets them quickly and efficiently identify therapy trends or behavior trends from therapy data and then communicate therapy or behavior recommendations to a patient.

Various embodiments disclosed herein relate to clinical decision support systems configured to automatically analyzing therapy data, identifying insights about a patient's therapy, and providing those insights to an HCP so that an HCP can make therapy and/or behavior recommendations to the patient in a timely, contextually relevant manner. In one embodiment, content for a recommendation based on insight may also be automatically provided to an HCP, and a user interface may be provided for reviewing insights, selecting content for recommendations, editing recommendations, and sending recommendations to a PWD.

Various embodiments described herein relate, generally, to systems and interfaces for HCPs to update therapy and behavioral recommendations for a PWD that are provided by clinical decision support systems. In various embodiments, the system and interface simplifies the workflows of HCPs by providing a preset list of insights to the HCP. In various embodiments, insights are trends that are observed using blood glucose data and/or therapy data, and which may be characterized, and/or translated into actionable and causational messages which are sent to a PWD. An HCP may use the interfaces disclosed herein to research insights, and determine which insights are relevant to send to a PWD. In some embodiments, the interfaces disclosed herein enable an HCP to receive or have access to a status of such insight being accepted or rejected by the PWD. In some embodiments, interfaces disclosed herein enable an HCP to access supporting data for insights highlighted within various reports to guide an HCP in clinical decision support. In some embodiments, parameters of insights may be configured by an HCP. For example, an HCP can revise an insight (by changing parameters of the insight) based, at least in part, on PWD feedback and/or motivation.

As already mentioned, various embodiments described herein relate, generally, to systems and methods for providing clinical decision support, and more specifically, to supporting HCPs in improving health outcomes for treatment of PWDs. Examples of improving health outcomes may include improving time within a target glucose range, reducing number and/or severity of episodes of hypoglycemia and diabetic ketoacidosis (DKA), and more generally, reducing risk of long-term complications. Examples of support for HCPs may include reducing time, effort, and/or expertise required by a provider to support a patient and/or therapy management systems. Additionally, support for HCPs may include standardization of care. For example, HPCs oftentimes interpret raw data differently and potentially lead to different therapy solutions. The preset insights will alleviate this issues and lead to a more consistent and standardized care for PWDs.

As used herein, the term “insight” means a recognition of a behavioral or therapy related trend or pattern that is clinically relevant to a PWD. One or more behavioral or therapy recommendations may be associated with an insight. Behavioral recommendations are recommendations for behaviors that, when implemented by a PWD, are associated with improved therapy outcomes for the PWD (e.g., improving time within a target glucose range, reducing number and/or severity of episodes of hypoglycemia and diabetic ketoacidosis (DKA), and more generally, reducing risk of long-term complications).

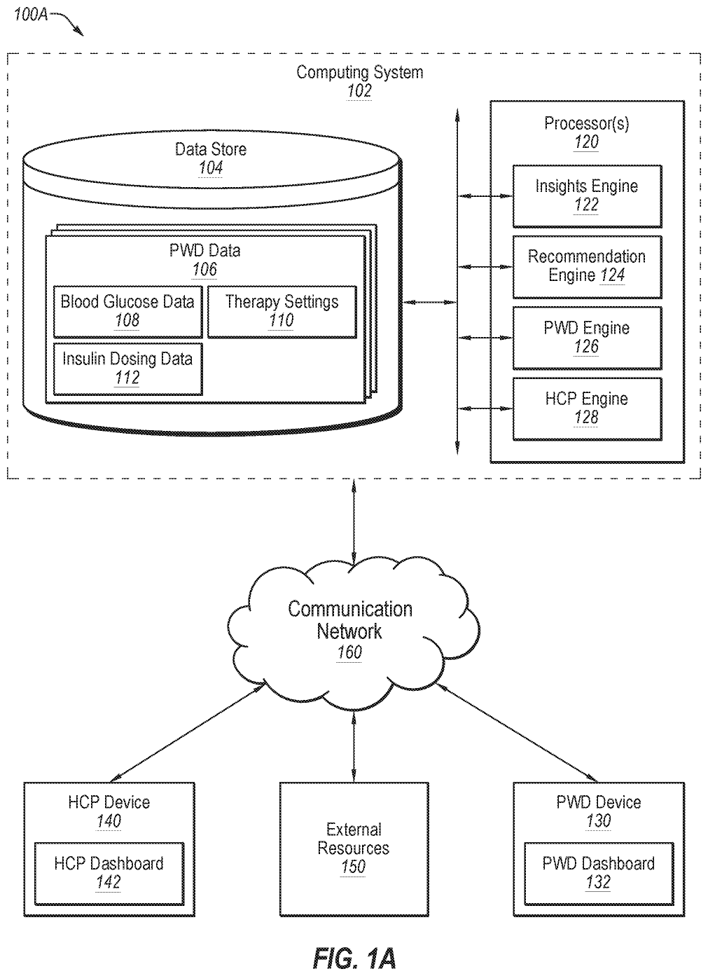

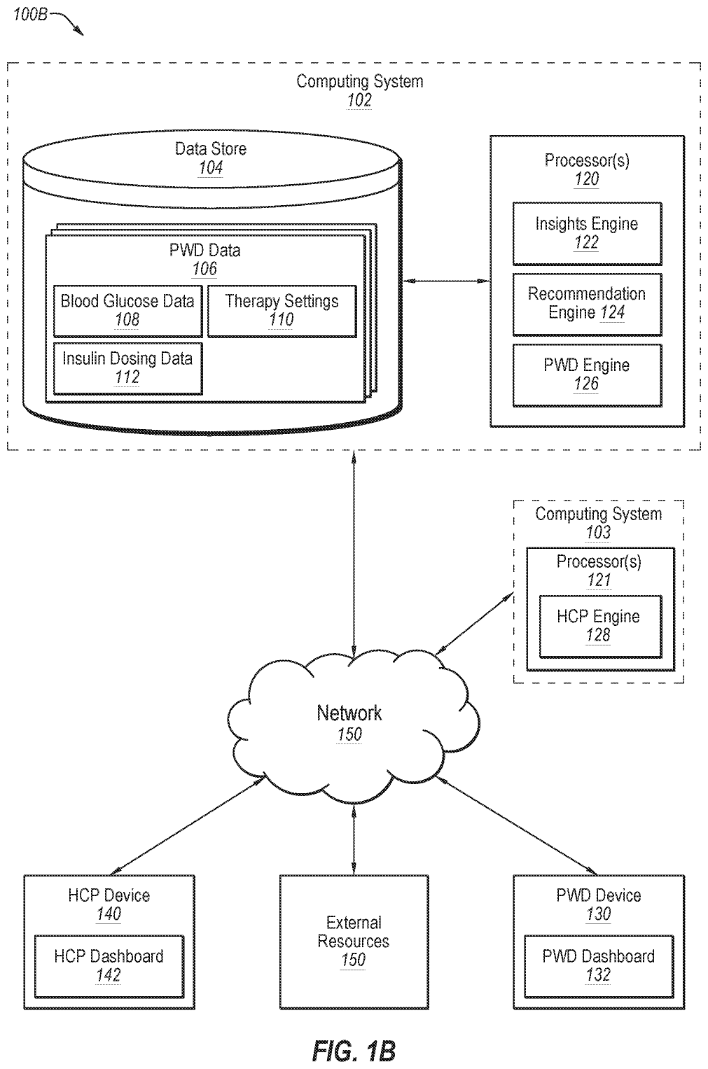

FIG. 1A illustrates a block diagram of a clinical decision support (CDS) system 100A, in accordance with one or more embodiments of the disclosure. In some embodiments, CDS system 100A may include one or more servers, and the servers may be configured to communicate with one or more client computing platforms according to a client/server architecture and/or other architectures. Client computing platform(s) may be configured to communicate with other client computing platforms via server(s) and/or according to a peer-to-peer architecture and/or other architectures. Users may also access CDS system 100A via client computing platform(s).

Embodiments of CDS system 100A, and methods of configuring and operating CDS system 100A, may be performed, in whole or in part, in cloud computing, client-server, or other networked environment, or any combination thereof. The components of such a system may be located in a singular “cloud” or network, or spread among many clouds or networks. End-user knowledge of a physical location and/or configuration of components of a system are not required.

In some embodiments, server(s), client computing platform(s), and/or various external resources may be operatively linked via one or more electronic communication links. For example, such electronic communication links may be established, at least in part, via a network such as the Internet and/or other networks. It will be appreciated that this is not intended to be limiting, and that the scope of this disclosure includes embodiments in which server(s), client computing platform(s), and/or external resources may be operatively linked via some other communication media.

A given client computing platform may include one or more processors configured to execute computer program modules. The computer program modules may be configured to enable an expert (e.g., an administrator) or user associated with a given client computing platform to interface with CDS system 100A and/or external resources, and/or provide other functionality attributed herein to client computing platform(s). By way of non-limiting example, a given client computing platform may include one or more of a desktop computer, a laptop computer, a handheld computer, a tablet computer, a NetBook, a smart phone, a gaming console, a media console, a set top box, a kiosk, and the like.

In one or more embodiments, CDS system 100A includes computing system 102, PWD device 130, and HCP device 140, which are configured to communicate with each other by way of communication network 160.

Computing system 102 includes data store 104. Data store 104 may be configured to store PWD data 106 (or insulin therapy data). Data store 104 includes one or more of blood glucose data 108, therapy settings 110 (or insulin therapy settings), and insulin dosing data 112. PWD data 106 may be received from external resources 150 (e.g., therapy management system, a therapy management application, blood glucose sensors, insulin delivery devices, and combinations thereof). In one or more embodiments, blood glucose data 108 may be raw blood glucose measurement, blood glucose estimates based on blood glucose measurements, and/or aggregations of the same, for example, trends or metrics. Blood glucose data 108 can include date, time and value of one or more measurements of blood glucose. Blood glucose data 108 may include timing information such as a time or time range associated with generating a blood glucose measurement from blood samples.

In various embodiments, blood glucose data 108 may be provided from any suitable glucose sensor. In some embodiments, a glucose sensor may be a continuous glucose monitor (CGM), a flash glucose monitor, a blood glucose meter (BGM), or any other suitable sensor. In the case of CGMs and flash glucose monitors, they may be configured to provide glucose data based on interstitial fluid glucose levels of a user, which may be correlated to blood glucose levels. A BGM may be configured to provide blood glucose data, typically based on a blood sample. Accordingly, the term “blood glucose” is not limited to using just blood glucose data, values, levels, etc., but is also intended to include interstitial fluid glucose levels, as well as any intermediate measurement values.

Therapy settings 110 may include information relevant to insulin for a PWD. Therapy settings 110 may include, for example, brand names of long acting and rapid insulin, number and types of insulin delivery devices used by a PWD, typical dose amounts of insulin for certain carbohydrate intake events (e.g., low carb meal, medium carb meal, high carb meal), and typical dose amounts of insulin before physiological events such as exercise. In some embodiments, therapy settings 110 may include individualized settings for a PWD, for example, a basal rate, a carbohydrate ratio, and/or an insulin sensitivity factor. For loop type delivery systems (e.g., artificial pancreas), therapy settings 110 may include correction settings, that is, settings related to providing correction doses of insulin. Additionally, therapy settings 110 may include correction information along with meal insulin for RA insulin delivery.

Insulin dosing data 112 may include dosing event information. Insulin dosing event information may include information about insulin dosing actions, for example a dosing time or time range, type of insulin (e.g., long acting (LA) insulin and rapid acting (RA) insulin), brand of insulin, and/or amount of dosed insulin. In some embodiments, dosing event information may include an indication of a dosing mechanism, for example, injection pen, inhaler, or infusion pump. Further, in some embodiments, dosing event information may include an indication of whether dosing event information, in part or in whole, is based on an actual dosing action (e.g., detecting insulin delivery, for example, based on a manual action of a pump or a control signal configured to cause insulin delivery), user tracking of dosing actions (e.g., a PWD or caregiver enters a dose using a therapy application executing on a mobile device), or inferred dosing actions (e.g., from capping/uncapping of an injection pen). Additionally, dosing event information may include removal of incorrectly inferred dosing actions (e.g., cap is removed and no dose was given).

Processors 120 may execute a number of engines for facilitating clinical decision support functions described herein. In one or more embodiments, such engines may include insights engine 122, recommendation engine 124, PWD engine 126, and HCP engine 128. More detailed description of PWD engine 126 and HCP engine 128 are provided in further detail below.

Insights engine 122 may be configured, generally, to recognize and acknowledge patterns (“pattern” is used herein to mean both patterns and trends, and legal equivalents thereof) within PWD data 106, and such patterns may relate to, for example, system and user behavior. In various embodiments, insights may be classified as behavioral insights, insulin dosing insights and positive insights (for PWD). In one or more embodiments, insights engine 122 may use rules and conditions to recognize an insight. Data, trends, and recommendations may be associated with insights for presentation to HCPs and/or PWDs. Recommendations engine 124 may be configured, generally, to determine recommendations for HCPs responsive to insights generated by insight engine 122.

CDS system 100A, in various embodiments, includes external resources 150. External resources 150 may include sources of information outside of CDS system 100A, external entities participating with CDS system 100A, and/or other resources. In some embodiments, some or all of the functionality attributed herein to external resources 150 may be provided by resources included in CDS system 100A. External resources 150, in one embodiment, may include medical devices. Medical devices may include insulin delivery systems, including without limitation, insulin delivery devices (e.g., infusion pumps, injection pens, and inhalers), glucose sensors (e.g., CGMs and blood glucose meters), therapy managers (e.g., controllers for controlling open and closed-loop delivery of insulin or aspects of delivering insulin and recommendation systems for providing therapy recommendations to users and/or health providers), and combinations thereof.

External resources 150, in various embodiments, may include a therapy management system(s), an example of which will be described in further detail below. Therapy management systems may include, among other things, a diabetes management system for checking blood glucose data and therapy data and managing therapy settings.

In some embodiments, one or more of PWD engine 126 and HCP engine 128 may be located in a separate computing system (physically or logically) from computing system 102. In the example shown in FIG. 1B, HCP engine 128 is located in computing system 103 for managing an HCP experience, and PWD engine 126 is located in a computing system 102 for managing a PWD experience. Insights and recommendations (or indications thereof) generated by insights engine 122 and/or recommendation engine 124 may be sent to PWD engine 126 and HCP engine 128, which manage the user experience at PWD dashboard 132 and HCP dashboard 142, respectively. For example, summaries of insights and available recommendations may be sent to HCP engine 128. The management (e.g., rules for) of how insights and recommendations are displayed and HCP input collected may be controlled by HCP engine 128. Selected recommendations and insights, as well as any changes by the HCP (e.g., additional content or a new recommendation) may be sent to computing system 102. Computing system 102 may then send the selected recommendations and insights and any changes to PWD engine 126, which manages the user experience for the PWD.

FIG. 1B depicts an embodiment of CDS system 100B. CDS system 100B is similar to CDS system 100A, however, CDS system 100B includes computing system 103 that is separate and distinct from computing system 102. In various embodiments, computing system 102 and computing system 103 operate, in whole or in part, in cloud computing, client-server, or other networked environment, or any combination thereof that are separate and distinct from another. Accordingly, at least PWD engine 126 operates in a computing environment that is separate and distinct from the computing environment associated with HCP engine 128. In various embodiments described herein, features and functionality of CDS system 100A are the same or at least similar to the features and functionality of CDS system 100A. As such, reference to features/functionality of CDS system 100A, herein, may also reference features/functionality of CDS system 100B.

CDS system 100A (and/or 100B) is configured, generally, to provide clinical decision support for HCPs in improving health outcomes for treating PWD. More specifically, insights engine 122 is configured to analyze PWD data 106, detect clinically relevant patterns, and detect therapy insights based on the detected patterns. Recommendations engine 124 is configured, for each insight identified by insights engine 122, to determine if a therapy recommendation is warranted, and if so, identifies one or more therapy recommendations to associate with the insight.

In one embodiment, insights engine 122 may be configured to select one or more therapy insights, including those therapy insights listed in Table 1, Table 2, and Table 3 provided below. Table 1 includes behavioral insights, Table 2 includes insulin dosing insights and Table 3 includes positive insights. Behavioral insights, insulin dosing insights, and positive insights all being types of therapy insights, as more fully described herein. It should be appreciated that disclosed embodiments are not limited to the insights listed in the tables provided herein, which are non-limiting examples of insights, one or more of which, may be used by embodiments of CDS system 100A. In the example contemplated by Tables 1, 2 and 3, a PWD is using a CGM to capture blood glucose data.

TABLE 1
Examples of behavioral insights

| | | | | | Rele- | |
|---|---|---|---|---|---|---|
| | | | Recommendations | | vant | |
| | | Data/Trend Presented to | viewed by HCP | | Set- | |
| Name | Rules/Logic to Trigger | HCP | (pushable to PWD) | Relevant Stat | tings | NPV Message |
| Wear | For the most recent | Low sensor usage: For the | Consider wearing | % of time with | N/A | Want to trade some |
| Sensor | 14 days, when | past 14 days, patient wore | CGM more frequently. | an Active CGM | | fingersticks for scans? |
| More | a CGM is active for | sensor <85% of the time. | Consider reviewing | out of 14 days | | CGM has benefits. |
| | <85% of the total | | this module on the | | | LINKS to LEARNING |
| | available time, then | | benefits of CGM | | | MODULES |
| | trigger an insight. | | Consider reviewing | | | View benefits of CGM |
| | Reference: | | this module on CGM | | | reusable learning |
| | JDRF RCT published | | troubleshooting | | | content |
| | in NEJM | | | | | View CGM |
| | | | | | | troubleshooting |
| | | | | | | reusable learning |
| | | | | | | content |
| Scan | For the time when | Too few scans: For | Consider scanning your | % of CGM data | N/A | A scan every |
| More | a CGM is Active | the past 14 days, when | sensor at least every | captured out of total | | 8 hours won't |
| | within the most recent | the patient was wearing | 8 hours to capture | possible CGM data | | hurt . . . but it can |
| | 14 days, if the total | a sensor, patient | as much CGM data as | available | | connect the dots in |
| | possible CGM | scanned too infrequently; | possible. It is especially | Avg number of | | your glucose. |
| | data points captured is | therefore, <90% of CGM | important to remember | scans/day | | LINKS to |
| | <90%, then trigger | data captured. | scanning before you go | | | LEARNING |
| | an insight. | | to bed and first thing in | | | MODULES |
| | | | the morning, to capture | | | View benefits |
| | | | your overnight glucose | | | of CGM reusable |
| | | | levels. Consider | | | learning content |
| | | | reviewing this module | | | View CGM |
| | | | on the benefits of CGM | | | troubleshooting |
| | | | Consider reviewing | | | reusable |
| | | | this module on | | | learning content |
| | | | CGM troubleshooting | | | |
| Check | For the most recent | Missed scan before RA | To get more informed | Avg number of | N/A | We missed an |
| Glucose | 14 days, count the | dose: Over the last | RA dose suggestions, | scans/day | | opportunity to |
| Before | number of RA doses | 14 days, patient took | consider scanning or | Avg number of | | support you . . . |
| Dosing | when: No CGM scan | X number of RA doses, | checking BG before | fingersticks/day | | scanning before |
| | or BG was taken | and for X of those doses, | taking a RA dose | Avg number of RA | | meals has benefits! |
| | 30 minutes prior | the patient did not scan | Consider reviewing this | doses/day | | LEARNING |
| | to the RA dose | sensor or check a BG | module on the benefits | | | MODULES |
| | | within 30-minutes | of CGM | | | View benefits |
| | | before taking the dose. | Consider reviewing this | | | of CGM reusable |
| | | | module on correction | | | learning content |
| | | | dose | | | View module |
| | | | | | | on correction |
| | | | | | | dose reusable |
| | | | | | | learning content |
| Upload | The RA and/or | Gaps in connectivity | Consider syncing your | Last time RA cap | | Consider syncing your |
| Data | LA caps have | between the caps | LA and/or RA caps with | was synced to MA. | | LA and/or RA caps |
| More | not been synced in. | and the MA: Patient's | your mobile app more | Last time LA cap | | with your mobile |
| Fre- | the last 14 days | LA and/or RA caps | frequently. Syncing your | was synced to MA. | | app more frequently. |
| quently | | have not been synced in | caps with the MA | | | Syncing your |
| | | 14 days. Insights cannot | generates useful glucose | | | caps with the MA |
| | | be generated without | and behavioral insights. | | | generates useful |
| | | the necessary data. | | | | glucose and |
| | | | | | | behavioral insights. |
| Rebound | For the most recent 14 | High after low: X times | Consider reviewing | Number of low | N/A | Pattern of high glucose |
| High | days, count the number | patient had a high | treatment of lows with | events in the last | | after a low glucose. [symbol] |
| | of highs (>180 mg/dL) | glucose event after | rule of 15. | 14 days. A low | | LEARNING |
| | [BGM or CGM value] | a low glucose event. | Consider reviewing this | event begins when | | MODULES |
| | that: Occur within | These events exclude | module on possible | glucose falls below | | View module on |
| | <3 hours of a preceding | periods of time when an | causes of low glucose, | 70 mg/dl and ends | | possible causes of low |
| | low (<70 mg/dL) | RA dose was given after | detection and | when glucose rises | | glucose, detection and |
| | RA dose not given | lows. | management | above 80 mg/dl. | | management reusable |
| | Note: Trigger if the | | | | | learning content |
| | fraction of lows | | | | | |
| | followed by rebound | | | | | |
| | highs is greater than 0.25. | | | | | |
| Dose for | For the most recent | Highs likely caused by | Consider having a | Avg number | | Pattern of missed |
| Every | 14 days, count the | missed meal doses: X times | reminder to take your | of RA doses/day | | meal doses. [symbol] |
| Meal | number of missed RA | patient had a high glucose | meal insulin for food | (over the last | | LEARNING |
| | doses using a missed | event likely caused by a | Contact me if you are | 14 days) | | MODULE |
| | dose detection algorithm, | missed meal dose. This was | still experiencing high | | | View module on |
| | Calculate the probability | observed through a high | glucose | | | possible causes |
| | of missing a dose as | glucose rate of change | Consider reviewing this | | | of high glucose, |
| | number of missed doses/ | with no RA dose. | module on possible | | | detection and |
| | (number of doses + | | causes of high glucose, | | | management reusable |
| | number of missed doses). | | detection and | | | learning content |
| | Trigger insight if the | | management | | | |
| | probability of a missed | | | | | |
| | dose is larger than 0.01. | | | | | |
| Dose | Count fraction of | Large time gap between | Consider taking your | | | Consider taking |
| Before | RA doses when: | eating and taking a rapid | rapid acting dose about | | | your rapid acting dose |
| Eating | Rate of change at the | acting dose: for x % of all | 15 minutes before you | | | about 15 minutes |
| | time of RA dose is | RA doses, the rate of | begin eating. This will | | | before you begin |
| | above 2 mg/dl/min | change when taking | reduce your after meal | | | eating. This will |
| | No previous RA dose | the dose was above | peak glucose and | | | reduce your after |
| | was taken in the last 2 | 2 mg/dl/min, suggesting | improve your overall | | | meal peak glucose |
| | hours | that the PWD started | time in range. | | | and improve your |
| | Glucose did not | eating before taking | | | | overall time in range. |
| | go below 70 mg/dl | their injection. | | | | LEARNING |
| | in the last 3 hours | | | | | MODULE |
| | Maximum glucose | | | | | View module on |
| | 0-4 hours after the RA | | | | | possible causes of high |
| | dose was above | | | | | glucose, detection and |
| | 180 mg/dl | | | | | management reusable |
| | Trigger if fraction of | | | | | learning content |
| | doses labeled as delayed | | | | | |
| | is greater than 0.25. | | | | | |
| Take LA | For the most recent | High caused by missed | Consider having a | | | It's a good habit |
| Dose | 14 days, count the | long acting doses: X LA | reminder to take your | | | to take your LA doses |
| Every | number of days when: | doses were missed or | long acting insulin dose | | | within 24 hours of |
| Day | The time between | taken too late in the | | | | each other. Consider |
| | two consecutive long | past 2 weeks; the | | | | having a reminder to |
| | acting does is more | patient had a high | | | | take your long |
| | than 24 hours | glucose event caused | | | | acting insulin dose |
| | Glucose rises above | by a missed LA dose. | | | | LEARNING |
| | 150 mg/dL (BGM or | | | | | MODULE |
| | CGM value) in the | | | | | View module on |
| | time period 24 hours | | | | | possible causes of high |
| | after the last LA dose | | | | | glucose, detection and |
| | until the next LA dose | | | | | management reusable |
| | and more than 3 hours | | | | | learning content |
| | after an RA dose | | | | | |
| | Glucose rises above | | | | | |
| | 250 mg/dL (BGM or | | | | | |
| | CGM value) in the | | | | | |
| | time period 24 hours | | | | | |
| | after the last LA dose | | | | | |
| | until the next LA dose | | | | | |
| | and within 3 hours of | | | | | |
| | an RA dose | | | | | |
| | Trigger if the number | | | | | |
| | of missed | | | | | |
| | LA doses in the last 2 | | | | | |
| | weeks is greater than 3. | | | | | |

TABLE 2
Examples of insulin dosing insights

| | | | | Rele- | Rele- | |
|---|---|---|---|---|---|---|
| | | | Recommendations | vant | vant | |
| | Rules/Logic | Data/Trend | viewed by HCP | Statis- | Set- | |
| Name | to Trigger | Presented to HCP | (pushable to PWD) | tics | tings | NPV Message |
| Needed | For the most recent | Need more insulin for | If fraction of times more | See | RA | You have either |
| more | 14 days, count the | meals: For days when a | insulin was needed | results/ | Dose | gone high or taken |
| insulin | number of times: | breakfast/lunch/dinner RA | is greater than 0.75: | logic | setting | a correction dose |
| for | the lowest glucose | dose was given, more insulin | “Consider increasing | trigger | | after your breakfast/ |
| meals | level 2-4 hours after | was needed X out of Y times | breakfast/lunch/dinner | | | lunch/dinner dose |
| | the meal dose was | (e.g. 5 out of 14 times). | RA dose by X units | | | X out of Y times. |
| | above 150 mg/dl, OR | Glucose was high after the | (10% rounded to 1 | | | LEARNING |
| | the lowest glucose | meal N out of M times and a | unit).” | | | MODULES |
| | level 2-4 hours after | correction dose was given O | If fraction of times more | | | View module on |
| | the meal dose was | out of P times. | insulin was needed | | | possible |
| | above 70 mg/dl, and | | is 0.25-0.75 times: | | | causes of highs |
| | a correction dose | | Consider reviewing this | | | View module on |
| | was given after the | | learning model on | | | corrections (reusable |
| | meal dose AND | | estimating meal size and | | | learning content) |
| | Glucose levels did not | | adjusting dose based on | | | |
| | fall below 70 mg/dl 0-4 | | meal size. | | | |
| | hours after the meal | | | | | |
| | dose | | | | | |
| | Only trigger if: | | | | | |
| | the “need less insulin” | | | | | |
| | after the same meal | | | | | |
| | insight is not triggered | | | | | |
| | the fraction of doses | | | | | |
| | needing more insulin | | | | | |
| | was greater than 0.25 | | | | | |
| | The “need to increase | | | | | |
| | LA dose” insight is not | | | | | |
| | triggered. | | | | | |
| | Whether an RA dose | | | | | |
| | corresponds to a small, | | | | | |
| | medium, or large meal is | | | | | |
| | determined by: | | | | | |
| | Calculating | | | | | |
| | recommended | | | | | |
| | correction dose at the | | | | | |
| | time of meals based on | | | | | |
| | PWD settings and | | | | | |
| | glucose at the time of | | | | | |
| | meal | | | | | |
| | Administered meal | | | | | |
| | dose = administered | | | | | |
| | dose − recommended | | | | | |
| | correction dose | | | | | |
| | Calculate | | | | | |
| | breakfast/lunch/dinner | | | | | |
| | meal dose | | | | | |
| | recommendations | | | | | |
| | based on PWD settings | | | | | |
| | Choose the category | | | | | |
| | where the absolute | | | | | |
| | difference between the | | | | | |
| | administered meal dose | | | | | |
| | and the category | | | | | |
| | dose is smallest | | | | | |
| | A correction dose | | | | | |
| | is a dose closer is | | | | | |
| | size to the estimated | | | | | |
| | correction dose | | | | | |
| | than to the estimated | | | | | |
| | correction | | | | | |
| | dose + small meal | | | | | |
| | dose recommendation. | | | | | |
| Needed | For the most recent | Need less insulin for meals: | If fraction of times less | See | RA | You have gone |
| less insulin | 14 days, count the | For days when a | insulin was needed is | results/ | dose | low X out of Y |
| for meals | number of times: | breakfast/lunch/dinner RA | above 0.25: Consider | logic | setting | times following a |
| | the lowest glucose | dose was given, less insulin | reducing your meal dose | trigger | | breakfast/lunch/ |
| | level 45 min-5 hours | was needed X out of Y times | X units (10% rounded to | | | dinner RA dose. |
| | after the meal dose was | (e.g. 5 out of 14 times). | by 1 unit). | | | LEARNING |
| | below 70 mg/dl, AND | | Consider | | | MODULES |
| | no correction dose was | | reducing your meal | | | View module on |
| | given after the meal | | dose after physical | | | possible causes of |
| | AND | | activity or in preparation | | | lows |
| | The “need to lower | | of physical activity | | | View module on |
| | LA dose” insight is not | | in the next 2 hours. | | | corrections (reusable |
| | triggered | | Alternatively, consider | | | learning content) |
| | Only trigger if fraction of | | eating a snack before | | | |
| | doses with low after the | | physical activity. | | | |
| | meal is greater than 0.25. | | | | | |
| Need | For the most recent | Need more insulin for | Consider increasing | See | Cor- | You have |
| more | 14 days, count the | corrections: For days when a | your morning/ | results/ | rection | remained high after |
| insulin for | fraction of RA doses | RA dose with correction was | afternoon/evening | logic | dose | correction doses |
| corrections | given when: the lowest | given, more insulin was | correction doses by X | trigger | settings | X out of Y times. |
| | glucose level 2-4 | needed X out of Y times. | (10% rounded to | | | LEARNING |
| | hours after a dose was | | the nearest 1 unit)”. | | | MODULES |
| | above 150 mg/dl, | | | | | View |
| | AND | | | | | module on possible |
| | glucose levels | | | | | |
| | did not fall below | | | | | causes of highs |
| | 70 mg/dl 0-4 hours | | | | | View module on |
| | after the correction | | | | | corrections (reusable |
| | dose AND | | | | | learning content) |
| | no additional | | | | | |
| | meal RA was given | | | | | |
| | 0-4 hours after the | | | | | |
| | initial dose | | | | | |
| | (A meal | | | | | |
| | RA is defined as an | | | | | |
| | RA where the dose | | | | | |
| | administered | | | | | |
| | was closer to the | | | | | |
| | recommended | | | | | |
| | small meal dose + | | | | | |
| | recommended | | | | | |
| | correction dose than | | | | | |
| | to the recommended | | | | | |
| | correction dose.) | | | | | |
| | Only triggered if: | | | | | |
| | number | | | | | |
| | of RA doses given | | | | | |
| | when glucose values | | | | | |
| | were above target | | | | | |
| | exceeded 5 | | | | | |
| | fraction of | | | | | |
| | correction doses where | | | | | |
| | more insulin was | | | | | |
| | needed exceeds 0.5 | | | | | |
| | “needed more insulin | | | | | |
| | after bf/lunch/dinner” | | | | | |
| | insight is not triggered | | | | | |
| | “Need more LA | | | | | |
| | insulin” insight is not | | | | | |
| | triggered | | | | | |
| | “need less insulin after | | | | | |
| | correction” in the same | | | | | |
| | time period is not | | | | | |
| | triggered | | | | | |
| Need less | For the most recent | Need less insulin for | Consider decreasing | See | Cor- | You have gone low |
| insulin for | 14 days, count the | corrections: For days when a | your | results/ | rection | after correction doses |
| corrections | fraction of RA doses | RA dose with correction was | morning/afternoon/ | logic | dose | X out of Y times. |
| | given when glucose | given in the | evening correction | trigger | settings | LEARNING |
| | values were above | morning/afternoon/evening, | doses by X (10% | | | MODULES |
| | target followed by: | less insulin was needed X out | rounded to | | | View module |
| | Glucose levels falling | of Y times. | the nearest 1 unit) | | | on possible |
| | below 70 mg/dl | | | | | causes of lows |
| | 45 min-5 hours | | | | | View module on |
| | after correction dose | | | | | corrections (reusable |
| | AND | | | | | learning content) |
| | no additional | | | | | |
| | correction RA was | | | | | |
| | given 0-4 hours after | | | | | |
| | the initial dose | | | | | |
| | Only triggered if: | | | | | |
| | number | | | | | |
| | of RA doses given | | | | | |
| | when glucose values | | | | | |
| | were above target | | | | | |
| | exceeded 5 | | | | | |
| | fraction | | | | | |
| | of correction doses | | | | | |
| | where less insulin | | | | | |
| | was needed | | | | | |
| | exceeds 0.25 | | | | | |
| | “needed | | | | | |
| | less insulin after bf/ | | | | | |
| | lunch/dinner” insight is | | | | | |
| | not triggered | | | | | |
| | The “need | | | | | |
| | to decrease LA dose” | | | | | |
| | insight is not triggered | | | | | |
| Need to | For the most recent | Low During Sleeping- | Consider reducing | X LA | LA | Pattern of lows |
| decrease | 14 days, count the | Hours: Patient experienced | LA insulin by | Doses | Dose | detected overnight/ |
| LA dose | number of days | lows during sleeping hours on | X units (10% rounded | Given/ | Settings | between meals |
| | when one or more lows: | X out of Y nights when an LA | to the nearest unit) | 14 days | | [symbol] |
| | Within 24 hours of | dose was given. | Consider reviewing this | | | LEARNING |
| | a LA dose | | module on possible | | | MODULES |
| | Overnight | | causes of low glucose, | | | View module on |
| | (where overnight is | | detection and | | | possible causes |
| | defined as the longest | | management. | | | of low glucose, |
| | period of time between | | | | | detection and |
| | 2 RA doses in | | | | | management reusable |
| | a 24-hour period) | | | | | learning content |
| | More than 4 hours after | | | | | |
| | the last RA dose | | | | | |
| | Note: | | | | | |
| | Trigger if: | | | | | |
| | Fraction of nights | | | | | |
| | with low | | | | | |
| | overnight | | | | | |
| | attributed to LA | | | | | |
| | dose exceeds 0.25 | | | | | |
| | Need more | | | | | |
| | insulin for meals | | | | | |
| | insight is | | | | | |
| | triggered | | | | | |
| | In this context, a low is | | | | | |
| | defined as a BGM | | | | | |
| | value below 70 mg/dl | | | | | |
| | or three consecutive | | | | | |
| | CGM values below 70 | | | | | |
| | mg/dl. | | | | | |
| Need to | For the most recent | Glucose rising during | Consider increase | X LA | LA | Pattern of high |
| increase | 14 days, count the | sleeping hours: For days | in long acting insulin by | Doses | Dose | glucose overnight [symbol] |
| LA dose | fraction of days when: | when a LA dose was given, | X units (10% rounded | Given/ | Settings | LEARNING |
| | Glucose values are | patient experienced X high | to the nearest unit) | 14 days | | MODULE |
| | increasing overnight | glucose events during | Consider reviewing this | | | View module on |
| | Overnight is | sleeping hours. | module on possible | | | possible causes |
| | defined by | | causes of high glucose, | | | of high glucose, |
| | identifying the | | detection and | | | detection and |
| | longest period of | | management | | | management reusable |
| | time between 2 RA | | Consider reviewing | | | learning content |
| | doses in a 24-hour | | material on snacks at | | | |
| | period), and | | bedtime and what | | | |
| | considering the | | relationship stress and | | | |
| | period of time 3 | | illness have on high | | | |
| | hours after the first | | glucose | | | |
| | RA dose until the | | | | | |
| | second RA dose | | | | | |
| | Increasing is | | | | | |
| | defined as glucose | | | | | |
| | at the beginning of | | | | | |
| | the time range | | | | | |
| | being at least 30 | | | | | |
| | mg/dl lower than | | | | | |
| | glucose before the | | | | | |
| | second RA dose | | | | | |
| | Glucose never drops | | | | | |
| | <100 mg/dL | | | | | |
| | Maximum overnight | | | | | |
| | glucose >130 mg/dl | | | | | |
| | Note: | | | | | |
| | Trigger only if fraction | | | | | |
| | of overnight highs | | | | | |
| | attributable to LA dose | | | | | |
| | exceeds 0.6. | | | | | |

For each behavioral insight and insulin dosing insight that insights engine 122 is configured to detect, insight engine 122 and/or recommendation engine 124 store an insight name, triggering logic, a template for explaining the data/trend that triggered the insight (and presentable to the HCP), a list of available behavioral recommendation(s), and a template for displaying relevant statistics(s). These fields correspond to columns headings for Table 1 and Table 2. For each positive insight that insights engine 122 is configured to detect, insights engine 122 stores an insight name, and rules that trigger it. These fields correspond to column headings for Table 3.

Behavioral insights are detectable by insights engine 122 from patterns in the PWD data 106. The patterns are pre-associated with behavioral insights. For example, insights engine 122 is configured to identify one or more of eight behavioral insights (i.e., detect patterns in the PWD data 106 associated with the behavioral insights) shown in Table 1: (1) wear sensor more; (2) scan CGM more, (3) check glucose before dosing; (4) upload data more frequently; (5) rebound high; (6) dose for every meal; (7) dose before eating; and (8) take a long acting dose every day.

Additionally or alternatively, insulin dosing insights are also detectable by insights engine 122 from patterns in the PWD data 106. The patterns are pre-associated with insulin dosing insights. For example, insights engine 122 is configured to identify one or more of six insulin dosing insights (i.e., detect patterns in the PWD data 106 associated with the behavioral insights) shown in Table 2: (1) needed more insulin for meals; (2) needed less insulin for meals; (3) need more insulin for corrections; (4) need less insulin for corrections; (5) need to decrease LA dose; and (6) need to increase LA dose.

Additionally or alternatively, positive insights are detectable by insights engine 122 from patterns in the PWD data 106. The patterns are pre-associated with positive insights. For example, insights engine is configured to identify one or more of twelve positive insights (i.e., detect patterns in the PWD data 106 associated with the behavioral insights) shown in Table 3: (1) best time in range day in the last month; (2) best time in range week in the last 3 months; (3) good glucose streak, in days; (4) good glucose streak, in weeks; (5) wearing your sensor more in the past 2 weeks; (6) scanning your sensor more in the past 2 weeks; (7) checking glucose before RA dose in the past 3 months; (8) thanks for uploading data; (9) good job dosing before meals; (10) remembering to take LA dose; (11) no rebound highs; and (12) changed RA or LA dose.

It should be appreciated that insights engine 122, in various embodiments, may comprise multiple insights engines. In one embodiment, insights engine 122 may include a behavioral insights engine, insulin dosing engine, and a positive insights engine. These insight engines may operate independently of each other, in other words, they may operate as independent processes within CDS system 100A, identifying insights.

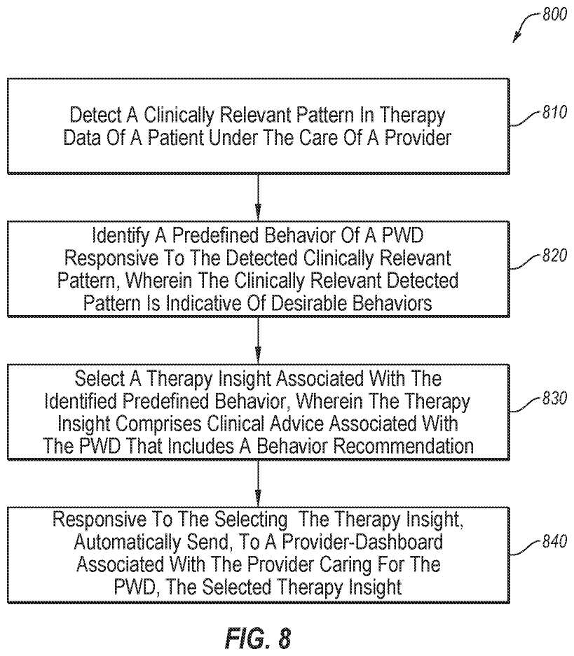

FIG. 8 illustrates a method 800 for automated provisioning of clinical advice to a provider caring for a PWD. Method 800, in various, embodiments, may be implemented by CDS system 100A.

At 810 of method 800, a clinically relevant pattern in therapy data of a PWD under the care of a provider is detected. For example, insights engine 122 receives PWD data 106 (e.g., therapy data) from data store 104. Upon receiving PWD data 106, insights engine 122 detects a clinically relevant pattern in the PWD data 106.

A clinically relevant pattern can be any pattern of data that is relevant to the therapy of a PWD. A clinically relevant pattern, in various embodiments, is a detected pattern in the PWD data 106 associated with the behavioral insights. For example, a clinically relevant pattern is a pattern listed in the “Relevant Statistics” column of Tables 1 and 2 (e.g., Table 1, Row 1, Column 5: % of time with an active CGM out of 14 days).

At 820 of method 800, a predefined behavior of a PWD is identified responsive to the detected clinically relevant pattern. For example, insights engine 122 identifies a predefined behavior of a PWD. A predefined behavior, in various embodiments, is a behavior described in “Data/Trend Presented to HCP” Column of Tables 1 and 2 (e.g., Table 1, Row 1, Column 3: Low Sensor Usage: For the past 14 days patient wore sensor <85% of time).

In various embodiments, the clinically relevant detected pattern is indicative of desirable behaviors and/or undesirable behaviors. An example of a detected pattern indicative of an undesirable behavior is the “low sensor usage” pattern (Table 1, row 1, column 3). The “low senor usage” pattern is indicative of an undesirable behavior because low usage of a CGM by a PWD does not enable the CGM provide a complete set of PWD data 106 for use by computing system 102.

An example of a detected pattern indicative of a desirable behavior are patterns in Table 3. In various embodiments, the patterns in the rules/logic to trigger column are indicative a desirable behaviors, such as the “time in range in the last day was better than in any other day in the last month” pattern associated with the “best time in range day in the last month” insight. The “time in range in the last day was better than in any other day in the last month” pattern is indicative of a desirable behavior because the increased time in range for a PWD improves health outcomes for the PWD.

At 830 of method 800, a therapy insight associated with the identified predefined behavior is selected. For example, insights engine 122 selects a “wear sensor more” insight (Table 1, row 1, column 1) associated with a “low sensor usage” pattern (Table 1, row 1, column 3).

In various embodiments, a therapy insight comprises clinical advice associated with the PWD that includes a behavior recommendation. Examples of behavior recommendations are provided in the “recommendations viewed by an HCP” column (e.g., Column 4) of Tables 1 and 2. In one example, the behavior recommendations for a “wear sensor more” insight includes (1) consider wearing a CGM more frequently, (2) consider reviewing a module on the benefits of CGM, and (3) consider reviewing a module on CGM troubleshooting.

At 840 of method 800, responsive to the selecting the therapy insight, the selected therapy insight automatically sent to a provider-dashboard associated with the provider caring for the PWD. For example, HCP engine 128 automatically transmits an insight (e.g., “wear sensor more” insight) to HCP device 140 in response to selection of the insight. Upon HCP device 140 receiving an insight, the insight is presented (e.g., displayed) on HCP dashboard 142.

In various embodiments, more than one insight is sent to the HCP device 140 where each insight includes at least one recommendation. For example, HCP engine 128 sends three separate insights to HCP device 140. Each of the insights includes an associated recommendation. As such, HCP engine 129 sends at least three separate recommendations to HCP device 140.

In one embodiment, a therapy insight, once created, includes clinical advice that includes one or more behavior recommendations. For example, a “wear sensor more” insight (in the first row of Table 1) includes behavior recommendations (listed in column 4 of Table 1). The behavior recommendations for a “not wearing sensor” insight includes the following recommendations: (1) consider wearing a CGM more frequently, (2) consider reviewing a module on the benefits of CGM, and (3) consider reviewing a module on CGM troubleshooting. The “scan more” insight (in the second row of Table 1) includes behavior recommendations (listed in column 4 of Table 1). The behavior recommendations for a “scan more” insight includes, among other things consider scanning your sensor at least every 8 hours to capture as much CGM data as possible.

In various embodiments, therapy insights may be positive or negative insights. A negative insight, for example, may correspond to patterns of behavior or insulin dosing that are correlated to decreased health benefits, or increased health risks or complications. For example, a “wear sensor more” insight (in the first row of Table 1) may be characterized as a negative insight. In such an example, a negative insight is based, at least in part on, therapy data that a PWD does not wear their sensor regularly, which is triggered when over a previous 14 day period a CGM was active less than 85% of the time.

A positive insight, in some embodiments, correspond to patterns of behavior that are correlated to increased health benefits or reduced health risks or complications. For example, a “best time in range day in the last month” insight (in the first row of Table 3) may be characterized as a positive insight. In such an example, a positive insight is based, at least, in part on, therapy data that a glucose level of a PWD is in range, for a time period, in the last day that was better than in any other day of the last month.

As described herein, insights engine 122 selects insights based, at least in part, on PWD data 106. In various embodiments, PWD data 106 used by insights engine 122 is current PWD data within a predetermined time frame (e.g., data from the most recent two weeks).

In various embodiments, therapy insights (that are selected by insights engine 122) are provided to HCP engine 128, which manages the HCP user experience at the HCP dashboard 142. For example, one or more insights (e.g., “not wearing sensor” insight) detected by insights engine 122 may be provided to HCP engine 128, which then provides therapy insights to the HCP dashboard 142, and then one or more therapy insights may be presented to an HCP on HCP dashboard.

In various embodiments, HCP engine 128 may be configured, generally, to generate system statistics; manage, send, and/or present therapy insights to an HCP; and receive HCP feedback, including without limitation, feedback about therapy insights presented at the HCP dashboard 142.

In some embodiments, HCP engine 128 may be configured to assign and re-assign priorities to therapy insights, which priorities may effect an order according to which therapy insights are presented (e.g. displayed) at HCP dashboard 142. By way of example, therapy insights corresponding to patterns of behavior that are more frequently recognized for a PWD may be presented above (e.g., in rank order) less frequently recognized insights. By way of another example, insights that are correlated with serious health risks, such as risk of hypoglycemia or risk of DKA, may be presented above insights less correlated with serious health risks. In one embodiment, therapy insights may be prioritized based, at least in part, on the impact to the PWD. An example prioritization, based on impact, may be LA insulin change, RA insulin change, behavioral improvement, and kudos for good behavior. In such an example, LA insulin change has a greater therapy impact to the PWD than a RA insulin change. Likewise, an RA insulin change has greater therapy impact to the PWD than a behavioral improvement and so on. It should be appreciated that prioritization of the insights may be customizable, for example, by a PWD and/or HCP.

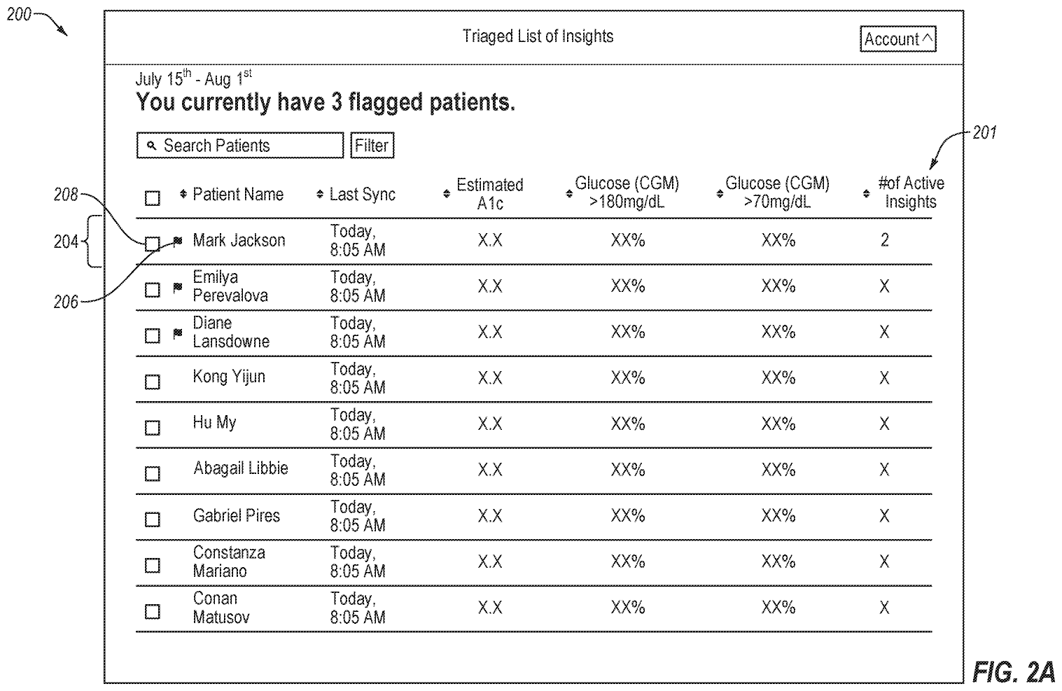

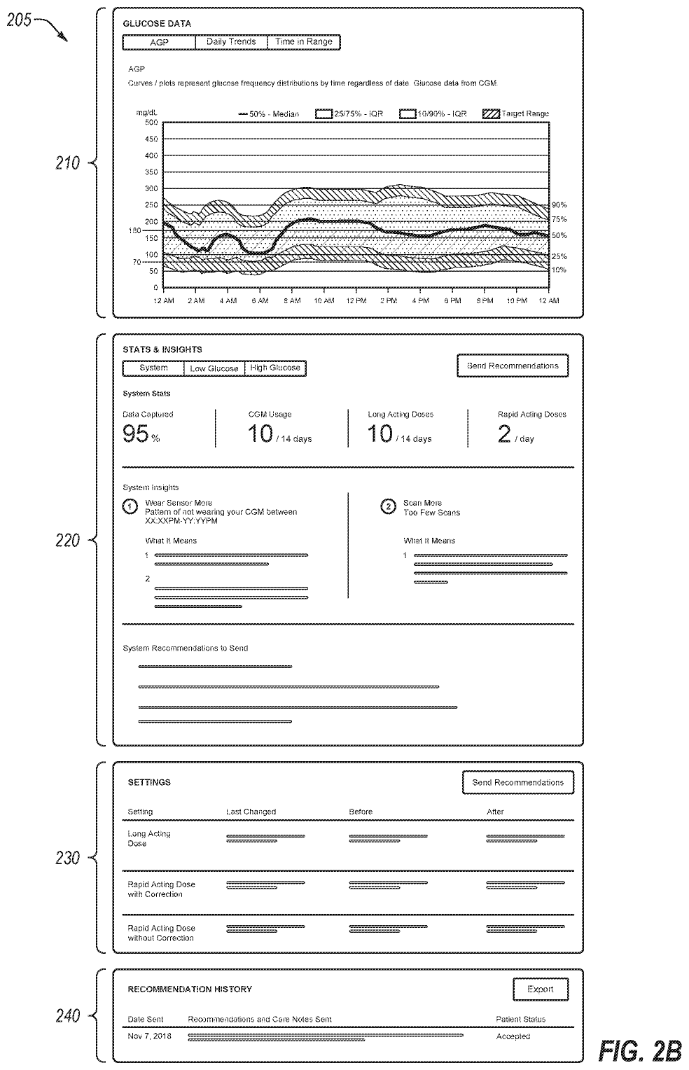

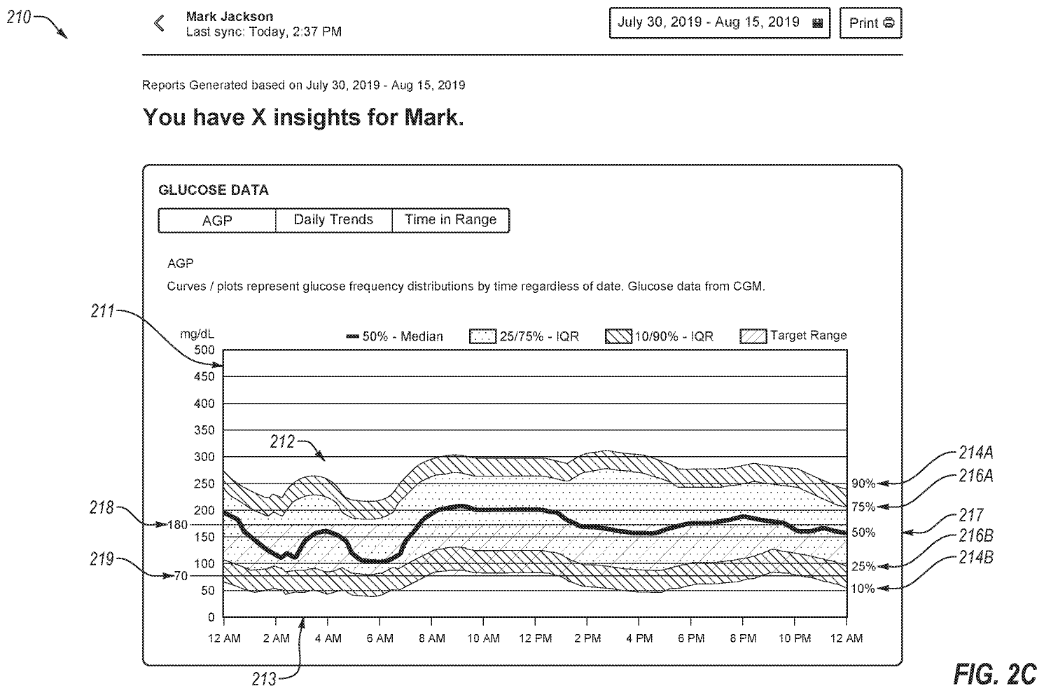

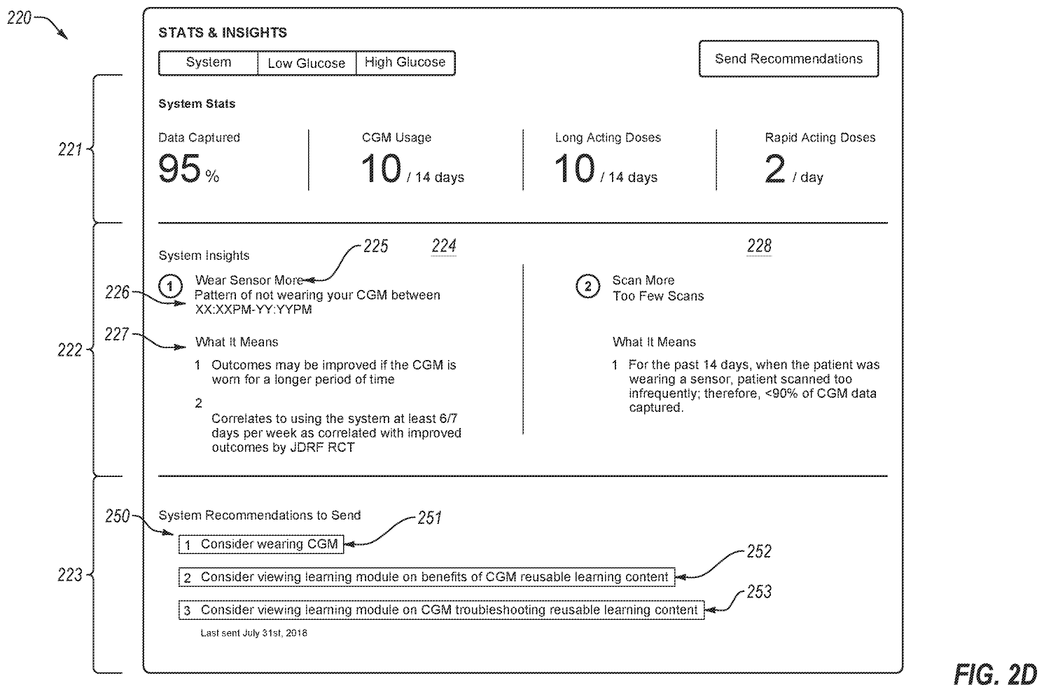

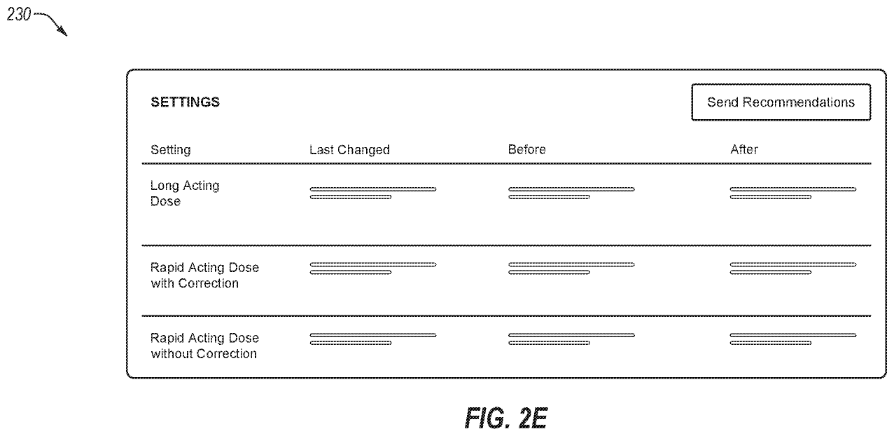

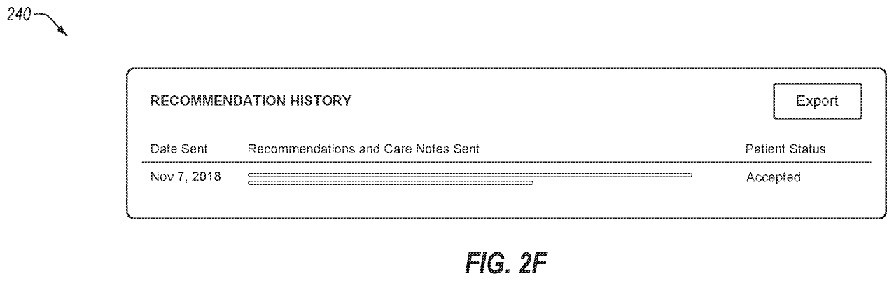

The therapy insight provided to an HCP support an HCP in improving outcomes for a PWD under the care of an HCP. Examples of improved outcomes include, but are not limited to, increased time within a target glucose range, reduced episodes of severe hypoglycemia, and reduced episodes of hyperglycemia. Further, as a result of such outcomes, a PWD's risk of long-term diabetic related complications is reduced. More specifically, if a PWD follows the recommendations that are provided by the system, that behavior is correlated to the improved health outcomes. So, improved outcomes can be attributed at least in part to information presented to an HCP on an HCP dashboard 142 as described with respect to FIGS. 2A-F.

FIG. 2A depicts an example triaged patient list 200 presentable to HCP dashboard 142, in accordance to various embodiments. An example patient 204, “Mark Jackson” is the first patient listed in patient list 200. It is noted that the patient list may be triaged based, at least in part, on insights (e.g., the number of insights associated with a patient). Flag 206 is presented and is an indicator that patient 204 may need attention. Also shown is some summary information about Mark Johnson's therapy, including estimated A1c (a picture of average blood glucose control for a given period of time), percentage of glucose measurements within (or outside) a target range, here, between 70 mg/dL and 180, a number of active insights.

Patient list 200 includes column 201 that indicates the number of active insights associated with a particular patient. As depicted in FIG. 2A, Mark Jackson has two active insights (as listed in column 201). For example, the two active insights are the wear sensor more insight (Table 1, row 1) and the scan more insight (Table 1, row 2).

In one embodiment, any number of boxes 208 (associated with a patient name) may be selected. Upon selection, a batch report of the selected patients is created. For example, a batch report may be printed.

In various embodiments, a patient in patient list 200 is selectable. For example, upon selection of a patient in patient list 200, summary view (or overview 205) may be presented at HCP dashboard 142, as shown in FIG. 2B. In various embodiments, a patient is selectable in patient list 200 by, hovering over the patient's name and selecting the patient in the list.

FIG. 2B depicts an embodiment of an overview 205 of an insight presented to an HCP on HCP dashboard 142. In one embodiment, an insight in overview 205 is associated with a PWD (e.g., Mark). It should be appreciated that overview 205 can include a list of insights (e.g., at least two insights) presented to an HCP on HCP dashboard 142 for an HCP. Overview 205 includes various sections associated with various components of an insight. For example, overview 205 includes the following sections: glucose data 210, statistics and insights 220, settings 230 and recommendation history 240. It should be appreciated that the order of sections 210-240 displayed on HCP dashboard 142 may be adjusted.

FIG. 2C depicts an embodiment of glucose data 210 (or glucose data section) of overview 205 of FIG. 2B. Glucose data 210 includes an example plot 211 of various glucose distributions over time. In one embodiment, plot 211 of glucose data (e.g., interstitial glucose data) is based at least in part on PWD data 106, such as glucose data. On the left side of plot 211, Y-axis 212 shows values for Mark Johnson's blood glucose levels over time in milligrams per deciliter (mg/dL), and such glucose levels are shown according to a linear scale, here, 50 mg/dL changes. On the right side of plot 211, X-axis 213 shows frequency distribution ranges over time. In this example, a 10/90 interquartile range (IQR) of blood glucose values is defined by upper dashed line 214A and lower dashed line 214B. A 25/75 IQR of blood glucose values is defined by upper solid line 216A and lower solid line 216B. A median for blood glucose values is shown by solid line 217. A target range for blood glucose values is defined between upper bound 218 (here 180 mg/dL) and lower bound 219 (here 70 mg/dL).

X-axis 213 shows times in one hour increments over a 24-hour time period. In one or more embodiments, the increments and time period may be settings and may be increased or decreased as needed. Additionally, plot 211 overlays glucose data of various days over a 24-hour time period

FIG. 2D depicts an embodiment of statistics and insights 220. In various embodiments, statistics and insights presented in overview 205 are generated by insights engine 122. Statistics and insights 220 includes three sections, system statistics 221, system insights 222, and system recommendations 223. System stats 221 may include data captures, CGM usage in days out of a time period, long acting doses during a time period, and number of rapid acting doses per day. For example, system stats 221 shows that 95% of data was captured, CGM was used 10 out of 14 days, 10 long acting does were detected over the 14 day period, and two rapid acting doses were given per day.

System insights 222 may include one or more insights for a patient, in accordance with one or more embodiments of the disclosure. Here, system insights 222 includes two primary insight regions 224 and 228, defined in part by a separator. Primary insight region 224 includes primary insight 225, here, “wear sensor more.” Primary insight region 224 also includes detected behavior statement 226, here “pattern of not wearing your CGM between XX:XXPM-YY:YYPM.” An associated time period during which the behavior was detected is provide with detected behavior statement 226, and, in some embodiments, such time period may be the same as the time period defined on X-axis 213 of plot 211 or be within the time period defined on X-axis 213 of plot 211. Providing a time period with statement 226 gives a user a convenient guidepost to associate at least part of plot 211 with statement 226. An HCP may compare data shown at the plot 211 with detected behavior statement 226 using the time period and, for example, decide if she agrees.

Primary insights region 224 also includes explanation 227, which includes one or more explanation messages about importance or implications of primary insight 225. Here, explanation 227 includes two explanation messages: first, “outcomes may be improved if the CGM is worn for a longer period of time;” and the second “correlates to using the system at least 6/7 days per week as correlated with improved outcomes by JDRF RCT.”

Similar to insight region 224, insight region 228 includes an insight statement (e.g., scan more), detected behavior statement (e.g., too few scans) and explanation (e.g., For the past 14 days, when the patient was wearing a sensor, patient scanned too infrequently; therefore, <90% of CGM data captured).

In various embodiments, any number of insights of Tables 1-3 can be presented in insights 222. For example, insights 222 can include any number of insights from Table 1, any number of insights from Table 2 and/or any number of insights from Table 3.

System recommendations 223 includes a recommendation list 250. In various embodiments, recommendations presented in overview 205 are generated by recommendation engine 124. Recommendation list 250 is presented to an HCP for review, editing, and/or approval. Upon approval, recommendation list 250 is provided to a PWD, for example, sent to PWD device 130 for display at PWD dashboard 132.

Recommendation list 250 includes recommendations 251, 252 and 253, the textual contents of which were generated by a clinical decision support system, such as CDS system 100A of FIG. 1A. In the example shown in FIG. 2D, recommendation 251 is “consider wearing CGM,” recommendation 252 is “Consider viewing learning module on benefits of CGM reusable content” and recommendation 253 is “Consider view learning module on CGM troubleshooting reusable learning content.”

In various embodiments, recommendations 251-253 are individually selectable (for review, editing and/or approval by an HCP), as indicated by the boxes surrounding each recommendation. For example, an HCP may select each of recommendations 251-253 to be sent to a PWD. In another example, an HCP may select a recommendation (e.g., recommendation 253) to be removed from recommendation list 250 such that the recommendation is not sent to a PWD. In a further example, an HCP may add a recommendation to recommendation list 250.

FIG. 2E depicts settings 230 of overview 205, in accordance with embodiments. Settings 230 may be configured, generally, to display therapy settings for a patient. In the example shown in FIG. 2E, therapy settings for long acting doses, rapid acting dose with correction, and rapid acting dose without correction are shown. Also shown is historical dosing information for each of the foregoing. The historical dosing information may indicate if there is a change to the therapy settings in response to an insight and what that change would be so an HCP can see the delta before insights are sent to a PWD. Additionally, historical dosing information may include initial first time or ongoing therapy settings changes that are independent from setting changes based on insights. In various embodiments, settings 230 may depict that the settings are updated by a system (e.g., loop system or auto-titration), by an HCP, or by a PWD.

FIC. 2F depicts recommendation history 240 of overview 205, in accordance various embodiments. Recommendation history 240 is a log of therapy suggestions approved to send to a patient. In the example shown in FIG. 2F, recommendation history 240 includes a number of entries, and for each entry may include a date sent, a summary or synopsis of recommendations and/or care notes sent that date, and a patient status, which is an indication of whether a patient accepted the recommendation and/or care notes (i.e., received and viewed it). The patient status may initially be set to an intermediate status, for example, “waiting” or “unviewed.” Additionally or alternatively, a patient status may be set as “pending” if a PWD has not seen a recommendation/care notes (e.g., there is no indication that a recommendation/care note has been presented at a PWD's device or dashboard) or “snoozed” a viewing of recommendations/care notes. In one embodiment, while a patient status is “pending,” an HCP can update or revise the pending recommendation. In various embodiments, when a patient views the related therapy suggestion at PWD dashboard 132, a “read” or “reviewed” message is sent, for example, to PWD engine 126 and/or HCP engine 128 and an update sent to HCP dashboard 142 to change patient status for an associated recommendation and/or care note for recommendation history.

In one embodiment, care notes are sent to a PWD. However, in various embodiments, care notes are not sent to a PWD.

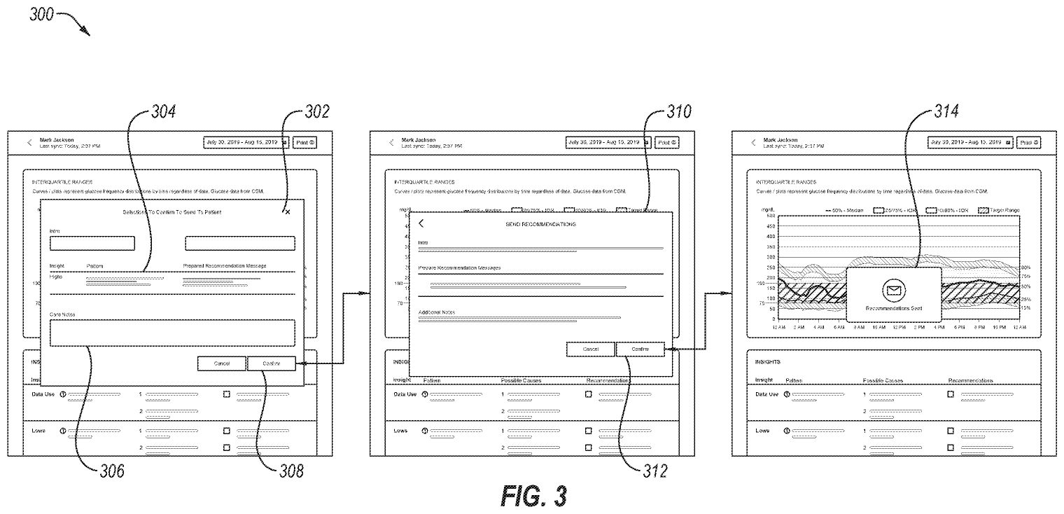

FIG. 3 depicts an example workflow 300 for selecting and sending recommendations to a PWD via HCP dashboard 142, in accordance with one or more embodiments of the disclosure. In the example workflow 300 shown in FIG. 3, a dialogue box 302 is displayed responsive to selection of one or more recommendations to send. For example, dialogue box 302 is displayed in response to an HCP selecting a recommendation (e.g., recommendation 251) in a list of recommendations (e.g., recommendations list 250) displayed on HCP dashboard 142.

Dialogue box 302 may include a summary 304 for each insight for which a recommendation message was selected. In this example, summary 304 includes insight description, a pattern description, and a proposed recommendation message. Dialogue box 302 also includes a text box 306 that may be used to enter notes, messages, or attach documents. For example, in response selecting a recommendation corresponding with an insight associated with a high glucose level, an HCP may enter information or messages for a PWD associated with the recommendation.

In some embodiments, content for a medically relevant note may be entered at text box 306. For example, the content may be part of an SOAP note (subjective, objective, assessment and plan note) that documents an aspect of a PWDs therapy.

Upon selection of button 308, “confirm” a confirmation request dialogue box 310 is displayed, which queries a user to confirm that the recommendations and/or care notes should be sent to the patient. In some embodiments, there may be an option to add an additional care notes (and/or revise care note in text box 306). Selection of button 312, “send” causes the recommendation to be sent to a PWD and for confirmation dialogue box 314 to be displayed with the message “recommendation sent.” In various embodiments, upon selection of button 312, the recommendation is sent to the PWD and/or content of a care note may be automatically saved to a care note of an electronic medical records (EMR).

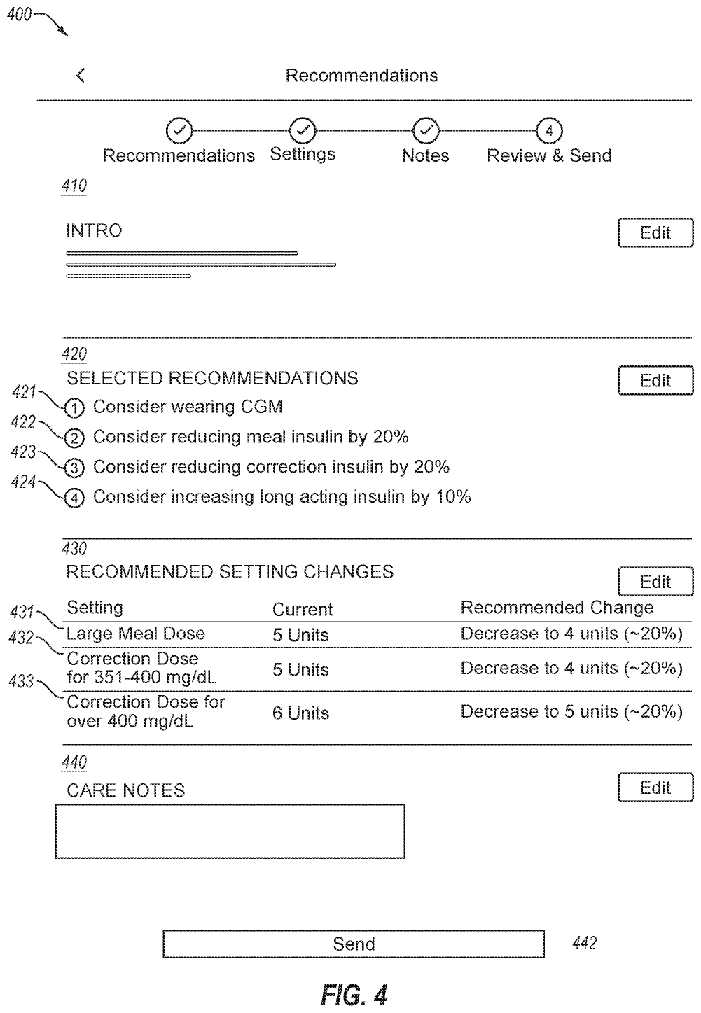

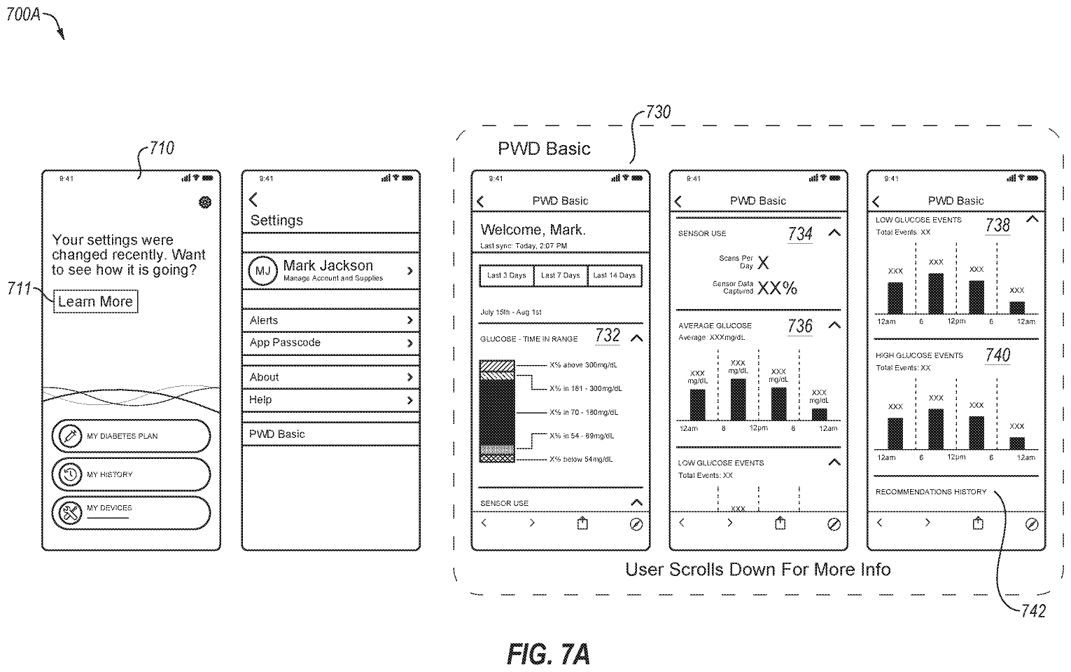

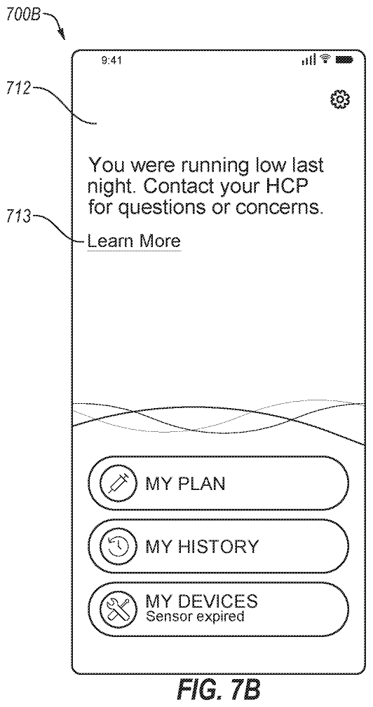

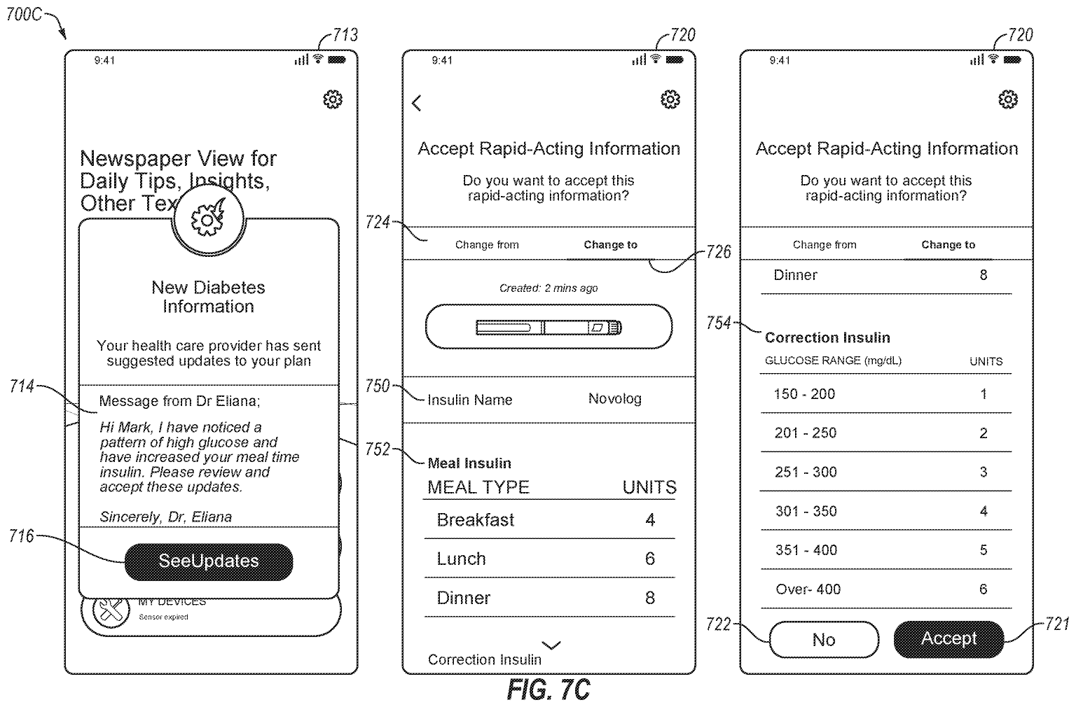

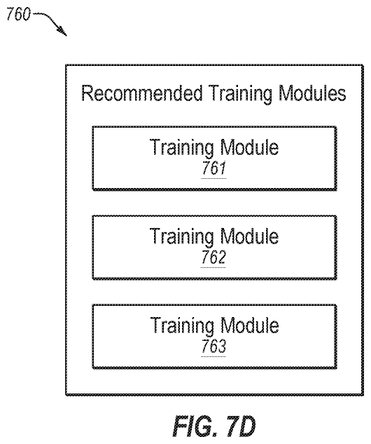

FIG. 4 depicts an example view 400 for displaying various information associated with recommendations, therapy changes and/or care notes using HCP dashboard 142, in accordance with one or more embodiments of the disclosure. View 400 includes various sections, such as, introduction 410, selected recommendations 420, recommended setting changes 430 and care notes 440. Introduction 410 allows an HCP to enter a personalized introduction (or message) to a PWD regarding selected recommendations 420 and/or recommendation setting changes 430. It is noted that introduction 410 is separate and distinct from care notes 440. For example, introduction 410 may include a personalized introduction (or overview) to the insights/recommendations, while care notes 440 include medically relevant information specifically associated with insights/recommendations. For example, an introduction may include the following message to the PWD: “Hi Mark, I have noticed a pattern of high glucose and have increased your meal time insulin. Please review and accept these updates. Sincerely, Dr. Eliana” (see FIG. 7C). In various embodiments, introduction 410 may be a preset message that is auto-compiled based, at least in part, on one or more insights/recommendations sent to a PWD. Alternatively, introduction 410 may be created by an HCP.

Selected recommendations 420 are one or more recommendations selected by an HCP to be sent to a PWD. As depicted in FIG. 4, selected recommendations 420 includes recommendation 421 (e.g., Consider wearing CGM recommendation), recommendation 422 (e.g., Consider reducing meal insulin by 20%), recommendation 423 (e.g., Consider reducing correction insulin by 20%), and recommendation 424 (e.g., Consider increasing long acting insulin by 10%). In various embodiments, recommendations may indicate percentage increase or decrease of insulin dosage, amount of units increase or decrease of insulin dosage, or simply directional increase/decrease of insulin therapy setting changes.

The list of recommendations may correspond to one or more insights (e.g., insights in Table 1, 2 and/or 3).

Recommended setting changes 430 (or adjusted insulin therapy settings) are one or more recommended therapy setting changes that may correspond to one or more of selected recommendations 420. Recommended setting changes 430 includes recommended setting change 431 to change a current dose (e.g., a large meal dose of insulin of 5 units) to an adjusted dose (e.g., 4 units), recommended setting change 432 to change a current dose (e.g., correction dose for 351-400 mg/dL of 5 units) to an adjusted dose (e.g., 4 units), and recommended setting change 433 to change a current dose (e.g., correction does for over 400 mg/DL of 6 units) to an adjusted dose (e.g., 5 units). In one embodiment, one or more of the recommended setting changes are accepted. As a result, the therapy settings are adjusted to the accepted recommended setting changes.

Care notes 440, in one embodiment, are similar to care notes entered into text box 306 (FIG. 3). Content of care notes 440 may comprise information and other documentation added, for example, by an HCP. Upon selection of send button 442, the information in introduction 410, selected recommendations 420, and recommended setting changes 430 are sent to a PWD. Content in care note 440 may also be sent to a PWD. Alternatively or in addition, in one embodiment, content of care notes 440, selected recommendations 420, and recommended setting changes 430 may be saved to an EMR system. In one embodiment, the content may be saved in fields for an SOAP note, or saved and referenced by an SOAP note reporting feature of an EMR system.

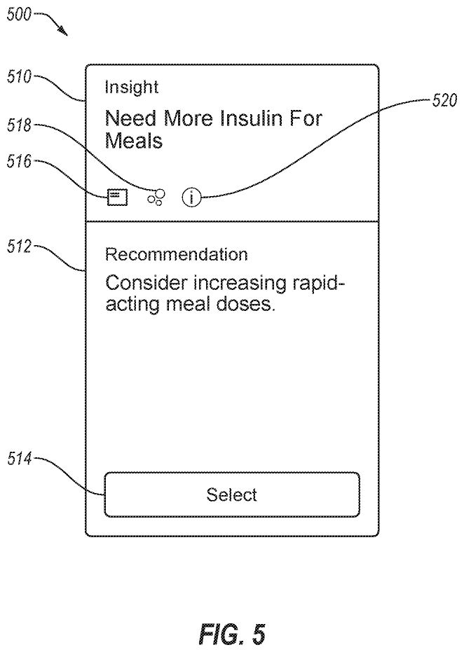

FIG. 5 depicts a view 500 of an insight displayed on HCP dashboard 142.

View 500 includes insight 510 (e.g., need more insulin for meals) and recommendation 512 (e.g., consider increasing rapid-acting meal doses) associated with insight 510. An HCP may approve or select the insight/recommendation to be presented to a PWD by selecting select button 514.

View 500 includes insight button 516, that when selected, view 500 is displayed. View 500 includes statistics button 518, that when selected, displays a statistics view associated with insight 510. View 500 includes information button 520, that when selected, displays information associated with insight 510. In various embodiments, the information displayed in response to selection of information button 520, may include, but is not limited to, information in any one of tables 1-3 (e.g., relevant stat, data/trend information).

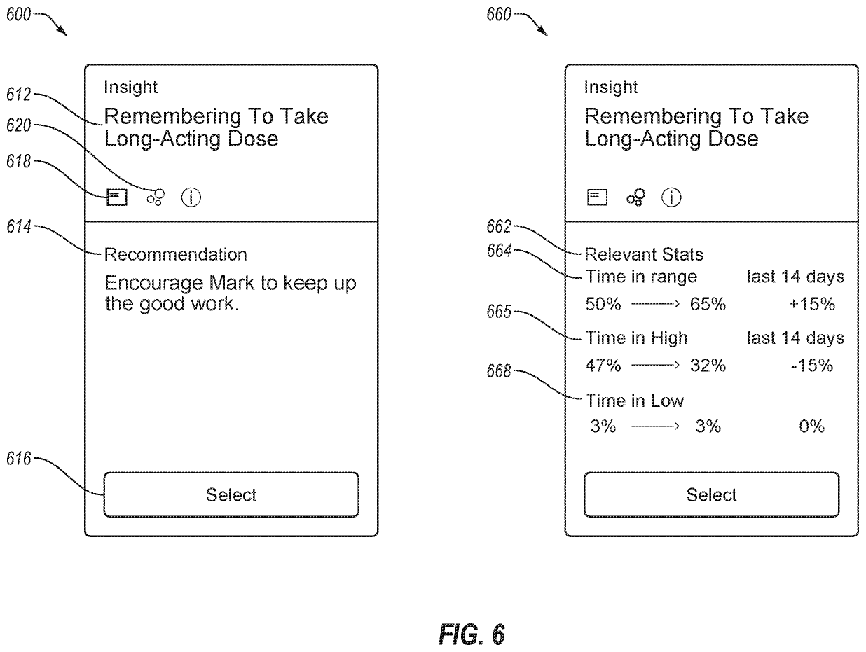

FIG. 6 depicts a view 600 of an insight displayed on HCP dashboard 142.

View 600 includes insight 612 (e.g., remembering to take long-acting dose) and recommendation 614 (e.g., encourage Mark to keep up the good work) associated with insight 612. An HCP may approve or select the insight/recommendation to be presented to a PWD by selecting select button 514.

View 600 includes insight button 618, that when selected, displays view 600. View 600 includes statistics button 620, that when selected, displays a statistics view (e.g., statistics view 660) associated with insight 612.

FIG. 6 also depicts view 660, which is a statistics view of insight 612, when statistics button 620 is selected. View 660 includes relevant statistics 662 associated with insight 612. For example, relevant statistics includes time in range 664, time in high 665 and time in low 668. It should be appreciated that relevant statistics can be any statistic associated with insight 612 (e.g., blood glucose level, time of active sensor, number of missed doses, etc.)

Time in range 664 depicts a 15% increase in the time in range in the past 14 days (e.g., from 50% to 65%). Time in high 665 depicts a 15% decrease of the duration of time in a high blood glucose range in the last 14 days (e.g., from 47% to 32%). Time in low 668 depicts a 0% change of the duration of time in a low blood glucose range in the last 14 days (e.g., no change from 3%).

FIG. 7A illustrates an example summary view 700A presented at PWD dashboard 132. Summary view 700A is configured, generally, to show behavior recommendations, therapy settings recommendations, and contextual information. Summary view 700A, in various embodiments, is displayed by selecting a request to look at updated information since receiving recommendations from an HCP, in accordance with one or more embodiments of the disclosure. For example, a PWD selects “learn more” button 711 (in view 710) of summary view 700A. Upon selection of “learn more” button 711, view 730 is presented at PWD dashboard 132. In the example view 730, shown in FIG. 7A, there are six sections, glucose 732, sensor use 734, average glucose 736, low glucose events 738, high glucose events 740, and recommendation history 742. In one embodiment, recommendation history 742 includes history of recommendations selected by an HCP and presented to a PWD. Recommendation history 742 may also include recommendations accepted/declined by a PWD. In various embodiments, the information depicted in view 730 depicts changes to therapy settings in view of insights and associated recommendations generated by CDS system 100A (and/or therapy management system 1000).

FIG. 7B illustrates an example view 700B presented at PWD dashboard 132. View 700B is configured, generally, to show an insight sent from an HCP. View 700B, in various embodiments, is displayed by selecting a request to look at updated information since receiving recommendations from an HCP, in accordance with one or more embodiments of the disclosure. As depicted, view 700B includes message 712 associated with an insight (e.g., blood glucose level is “running low last night”). Message 712, in various embodiments, may include real-time information. For example, message 712 includes real-time glucose levels generated by a CGM.

In one embodiment, a PWD selects “learn more” button 713. In various embodiments, upon selection of “learn more” button 713, an additional view (e.g., view 730) is presented at PWD dashboard 132 associated with the message 712. Alternatively, upon selection of “learn more” button 713, view 760 (in FIG. 7D) is displayed that depicts a list of selectable training modules associated with an insight.

FIG. 7C illustrates an example summary view 700C presented at PWD dashboard 132. Summary view 700C is configured, generally, to show views at a PWD dashboard of therapy recommendations received from an HCP. Summary view 700C includes message 714. Message 714 includes contextual information associated with an insight (e.g., “. . . pattern of high glucose . . . ”). In one embodiment, message 714 is an introduction message (e.g., introduction 410) drafted/edited by an HCP.

Upon selection of button 716 (e.g., “See Updates” button), view 720 is displayed. View 720 is provides information related to a contextual information in message 714. For example, view 720 provides information related to RA insulin settings. In particular, view 720 provides information related to recommended therapy setting changes associated with RA insulin.

In various embodiments, view 720 includes insulin name 750, meal insulin section 752, and correction insulin section 754.

Insulin name 750 indicates the particular RA insulin used by the PWD (e.g., Novolog). Meal insulin section 752 provides a list of meal types (e.g., breakfast, lunch, dinner) and the associated insulin units for each meal type. As depicted, recommended changes to RA insulin therapy includes changes to insulin units associated with the meal type. For example, the recommended therapy change for RA insulin is 4 units for breakfast, 6 units for lunch and 8 units for dinner.

Correction insulin section 754 provides a list of insulin units associated with glucose ranges. For example, the recommended therapy change for RA insulin is 1 unit for a glucose range of 150-200 (mg/dL), 2 units for a glucose range of 201-250, and so on.

View 720 includes “change to” button 726 and “change from” button 724. Upon selection of button 724, the current RA insulin setting are displayed. Upon selection of button 726, the recommended therapy changes to RA insulin settings are displayed.

View 720 includes accept button 721 and reject (or no) button 722. Upon selection of button 721, the recommended therapy changes to RA insulin settings (as depicted) are accepted. In one embodiment, the changes are automatically implemented. For example, accepted changes are implemented by therapy management system 1000 (which will be described in further detail below). In another embodiment, upon acceptance of therapy change recommendations, an indication of the acceptance by the PWD is transmitted to HCP device 140 for display on HCP dashboard 142.

Upon selection of button 722, the recommended therapy changes to the RA insulin settings are not accepted (or canceled). In another embodiment, upon selection of button 722, an indication of the PWD not accepting the recommended therapy changes is transmitted to HCP device 140 for display on HCP dashboard 142.

In various embodiments, display of view 720 is a basic or standard view of statistics associated with insights/recommendations. A PWD may desire more specific information regarding statistics associated with insights/recommendations. For example, summary view 700A, in FIG. 7A, depicts additional and more specific statistics associated with insights/recommendations as compared to view 720.

In various embodiments, care notes are presented in summary view 700C. For example, care notes are presented in view 720 or in another view not shown. The care notes can be created in a rich field text box (not shown) by a PWD. Alternatively, care notes are created by an HCP and presented at PWD dashboard 132.

FIG. 7D illustrates an example view 760 of recommended training modules presented at PWD dashboard 132. In various embodiments, view 760 is presented to a PWD in response to selection of a “learn more” button (e.g., “learn more” button 713, in FIG. 7B). View 760 includes one more learning modules associated with a recommendation. For example, view 760 depicts three separate (and selectable) training modules (e.g., training module 761, training module 762, and training module 763). However, in various embodiments, view 760 can include any number of selectable training modules.

The selectable training modules can be in various formats such as a video, document, audio, etc. For example, in response to selection of training module 761, a video is played to train a PWD. Training modules can include, but is not limited to, (1) benefits of CGM reusable learning content, (2) view CGM troubleshooting reusable learning content and the like.

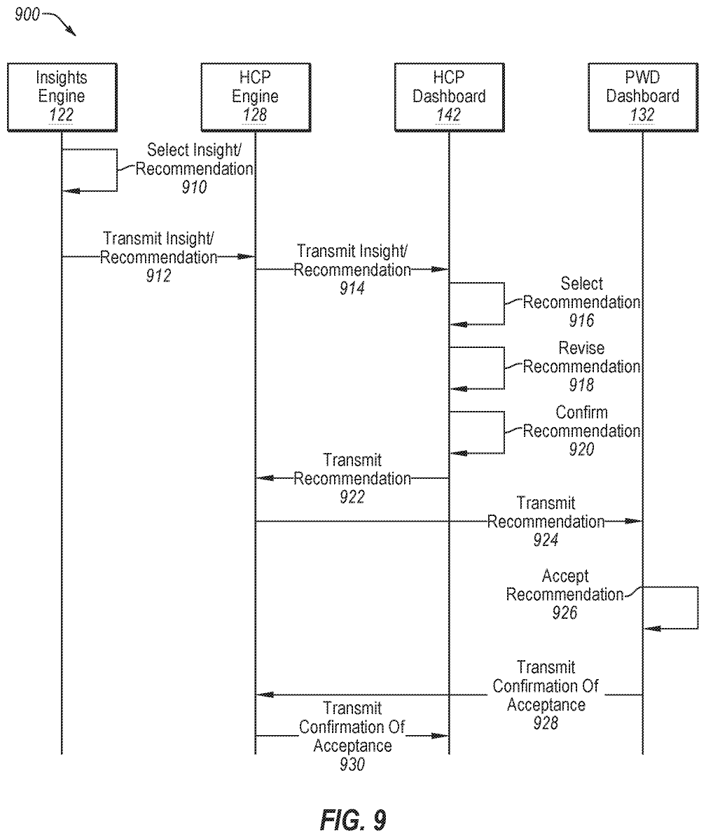

FIG. 9 depicts a method 900 of accepting a recommendation by a PWD. At operation 910, insights engine 122 selects a therapy insight associated with the identified predefined behavior. For example, insights engine 902 selects “wear sensor more” insight (Table 1, row 1). The “wear sensor more” insight includes one or more recommendations, such as, but not limited to, (1) consider wearing CGM more frequently, (2) consider reviewing a module on the benefits of CGM, and (3) consider reviewing a module on CGM troubleshooting

At operation 912, insights engine 122 transmits the insight and corresponding recommendation to HCP engine 128. At operation 914, HCP engine 129 transmits the insight and corresponding recommendation to HCP device to be presented on HCP dashboard 142.

At operation 916, an HCP selects one or more recommendations associated with an insight. For example, an HCP selects each of the recommendations associated with the insight (e.g., (1) consider wearing CGM more frequently, (2) consider reviewing a module on the benefits of CGM, and (3) consider reviewing a module on CGM troubleshooting). In various embodiments, the HCP can select a subset or none of the recommendations associated with an insight.

At operation 918, an HCP revises a recommendation. For example, an HCP adds care notes (e.g., care notes 440) to the selected recommendation. In various embodiments, an HCP is able to revise a recommendation (already sent to a PWD) when a PWD has not yet accepted or rejected a rejected the recommendation. At operation 920, an HCP confirms revision of the recommendation (e.g., confirms recommendation with added care notes 440).

At operation 922, the confirmed recommendation is transmitted from HCP dashboard 142 to HCP engine 128. At operation 924, recommendations (e.g., consider increasing meal dose or changing meal type and monitor glucose ˜2 hours after correction insulin doses delivered, etc.) are then transmitted to PWD device 130 and displayed on PWD dashboard 132. At operation 926, recommendations (e.g., consider increasing meal dose or changing meal type and monitor glucose ˜2 hours after correction insulin doses delivered, etc.) are selected and accepted by a PWD.

At operation 928, a confirmation of acceptance of recommendation by a PWD is transmitted to HCP engine 128. At operation 930, the confirmation is then transmitted to HCP device 140 and presented at HCP dashboard 142.

Some embodiments are directed to a clinical decision support system that operates in conjunction with a therapy management system and vice versa. In general, a therapy management system assists with managing insulin therapy for a PWD (or insulin-based management). For example, aspects of a therapy management system may control delivery of insulin to a patient such as a closed loop insulin delivery system, other aspects may manage settings and/or parameters for such a closed loop insulin delivery system, and other aspects still may include tools for collecting meal and exercise information from a PWD.

Therapy insights for a patient as well as recommendations selected by an HCP for a patient may be relevant to the operation of a therapy management system. For example, information that a CGM is active <85% of the time or an actual CGM active statistic (e.g., CGM only active 65% of the time) may be relevant to calculations of active insulin available that are based on glucose level data captured by a CGM.

So, in some embodiments, a therapy management system may be configured to receive therapy insights and/or recommendations and to assist with managing insulin therapy, at least, based in part, on insights and/or recommendations generated by CDS system 100A.

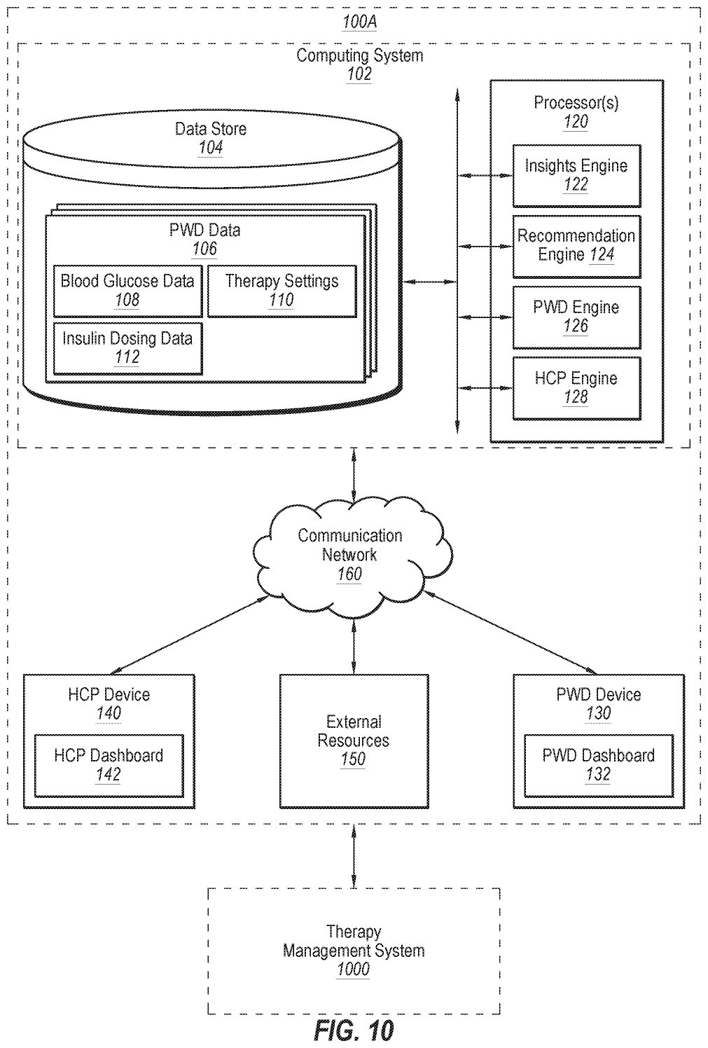

FIG. 10 depicts an embodiment of therapy management system 1000 communicatively coupled with CDS system 100. Therapy management system 1000 may be part of CDS system 100A, or it may be separate. In some cases, it is expected that there will be some overlap between elements of therapy management system 1000 and CDS system 100A. For example, PWD device 130 may run an application for PWD dashboard 132 and run a therapy management application (not shown). Additionally, therapy management system 1000 may communicate with CDS system 100A (or any device/engine of CDS system 100A) via communication network 160.

Therapy management system 1000, in various embodiments, may be configured, generally, to assist with managing insulin therapy for a patient. More specifically, therapy management system 1000 is configured to provide information about insulin therapy and provide therapy recommendations based on blood glucose data and insulin dosing data for a patient. In a case of manual delivery systems and open-loop delivery systems, therapy management system 1000 may be configured primarily to provide information and recommendations to a patient. In a case of closed-loop delivery systems, therapy management system 1000 may be configured to automatically adjust some therapy settings and user parameters (e.g., insulin sensitivity) based on blood glucose data, insulin dosing data, physiological data, and more.

In some embodiments, therapy management system 1000 may be configured to provide (e.g., report) a patient's therapy data to CDS system 100A that CDS system 100A utilizes to provide therapy insights and therapy recommendations. For example, referring to FIG. 10, therapy management system 1000 may be one of the external resources 150.

Therapy management system 1000 may receive or have access to some or all of the insights and/or recommendations provided by CDS system 100A based on the therapy data of PWD 1105. In one embodiment, only the therapy insights related to managing insulin therapy might be provided to therapy management system 1000, for example, an insight that a PWD did not wear her CGM during a certain time period. In another embodiment, only recommendations for specific therapy settings or therapy changes are sent to therapy management system 1000.

For example, referring to FIG. 4, recommendations 420 (selected by an HCP and sent to a PWD) are associated with recommended setting changes 430. Recommended setting changes 430 may be sent to therapy management system 1000, and therapy management system 1000 may, based on its settings, determine whether to change a current therapy settings based on one or more of recommended setting changes 430. In one embodiment, therapy management system 1000 may simply reject the recommendation because a discretionary setting at the therapy management system 1000 is set too low to allow the therapy management system 1000 to change therapy settings in general or to change the specific therapy setting. Alternatively or in addition, therapy management system 1000 may model glucose response for a PWD if the therapy setting is changed based on the recommendation, and then determine whether to change the setting based on the modeled glucose response of the patient.

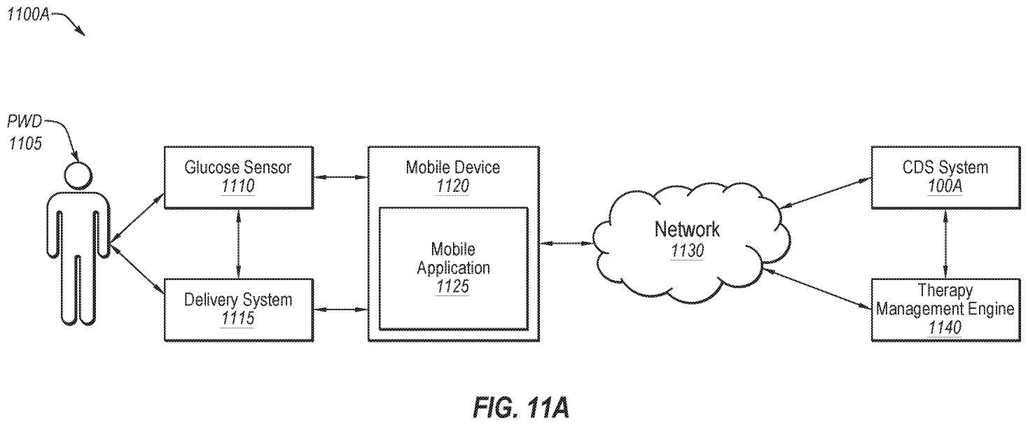

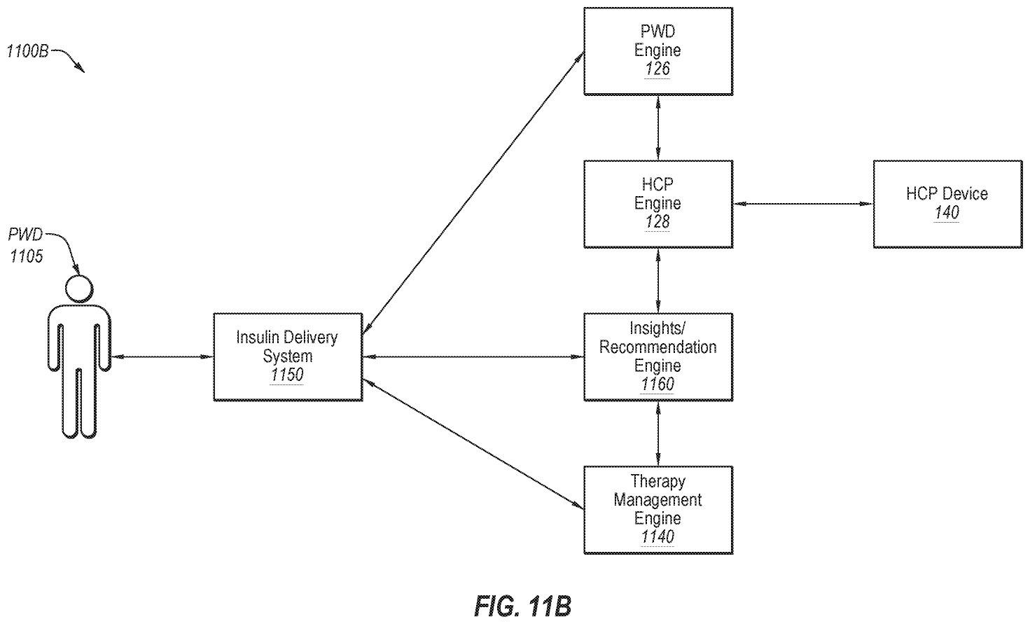

FIG. 11A depicts an embodiment of therapy management system 1100A. Therapy management system 1100 includes a glucose sensor 1110, delivery system 1115 and mobile device 1120.

Glucose sensor 1110 may be any suitable glucose sensor system, such as a blood glucose meter (BGM) adapted to determine blood glucose values using blood glucose test strips, and flash glucose monitor, or a CGM. In some cases, glucose sensor 1110 may be configured to act as both a flash glucose monitor and a continuous glucose monitor by permitting both intermittent and on-demand transmissions of blood glucose data. In some embodiments, glucose sensor 1110 can wirelessly transmit data when interrogated by a reader device (e.g., using NFC communication). In some embodiments, glucose sensor 1110 can wirelessly transmit data at predetermined intervals (e.g., using radio frequencies) using any suitable communication standard (e.g., Bluetooth Low Energy (BLE)). In some cases, systems and methods provided herein can include multiple glucose sensor systems (e.g., a continuous or flash glucose monitor and a blood glucose meter).

In some embodiments, glucose sensor 1110 can transmit glucose data using multiple communication techniques. In some embodiments, mobile device 1120 and/or delivery system 1115 may include an NFC reader adapted to obtain blood glucose data glucose sensor 1110 when brought within an interrogation distance of glucose sensor 1110. In some embodiments, glucose sensor 1110 broadcasts blood glucose data at predetermined periods of time (e.g., every 30 seconds, every minute, every 2 minutes, every 3 minutes, every 5 minutes, every 10 minutes, every 15 minutes, etc.).

In a polled (or interrogated) mode of operation, glucose sensor 1110 may wirelessly send blood glucose data to one or more of mobile device 1120 and delivery system 1115 that corresponds to a historical period. For example, when glucose sensor 1110 is interrogated, glucose sensor 1110 may send stored glucose data from the previous 1 hour, 2 hours, 3, hours, 4 hours, 5 hours, 6 hours, 7 hours, 8 hours, etc. In some cases, broadcast blood glucose data may only include a current or more recent blood glucose value. For example, in some cases blood glucose data may include only the most current readings (e.g., from the last 10 minutes).

Glucose sensor 1110 can transmit glucose data to CDS system 100A and stored in data store 104, as described herein. In one embodiment, glucose sensor 1110 transmits glucose data to mobile application 1125 of mobile device 1120. Upon receipt, mobile application 1125 transmits the glucose data (e.g., blood glucose data 108) to data store 104 of CDS system 100A via network 1130. In one embodiment, network 1130 is the same as network 160.

Mobile application 1125, in various embodiments, may execute on any suitable mobile computing device that can store and execute a mobile application that is adapted to display and input therapy relevant information wireless sly received from the other components of the system as well as from a graphical user interface that enables user to interact with the application. In one embodiment, mobile device 1120 can also store and execute a trusted mobile application within a trusted execution environment (hardware and/or software) that is not, generally speaking, accessible to users or devices communicating with mobile device 1120 but that is accessible to other applications executing on mobile device 1120. Various functions and calculations that relate to the therapy management system, including alerts and recommendations that are presented to users may be, in part or in whole, performed by the trusted mobile application. Moreover, some or all communication with delivery system 1115, such as, but not limited to, delivery system 1115, glucose sensor 1110, and, and other accessories may be restricted to the trusted mobile application.

As described herein, a PWD receives recommendations from an HCP. The recommendations may be displayed on a PWD dashboard (e.g., PWD dashboard 132) of mobile device 1120 via mobile application 1125. Upon acceptance of a recommendation, therapy management engine 1140 identifies one or more recommended therapy settings that are associated with a selected recommendation.

In one embodiment, therapy management engine 1140 automatically adjusts a therapy setting with the identified recommended therapy setting. For example, therapy management engine 1140 adjusts a current therapy setting of a large meal dose of insulin of 5 units to a large meal dose of insulin of 4 units.

Therapy management engine 1140 transmits the adjusted therapy setting to mobile application 1125 of mobile device 1120. In one embodiment, responsive to receiving the adjusted therapy setting, mobile application 1125 automatically implements the adjusted therapy setting. For example, mobile application 1125 provides instructions to delivery system 1115 such that delivery system 1115 implements the adjusted therapy setting.

In one embodiment, upon automatic implementation of the adjusted therapy setting, mobile application 1125 displays an indication to a PWD that a therapy setting has been changed to the adjusted therapy setting. In another embodiment, mobile application 1125 does not indicate to a PWD that a therapy setting has been changed.

In another embodiment, mobile application 1125 displays the adjusted therapy setting for acceptance by a PWD. For example, mobile application 1125 prompts a PWD to accept the adjusted therapy setting. Upon acceptance, mobile application implements the adjusted therapy setting, as described above.

FIG. 11B depicts an embodiment of therapy management system 1100B. Therapy management system 1100B includes insulin delivery system 1150. In various embodiments, insulin delivery system 1150 includes is an open loop system or a closed loop system. An open loop system involves a PWD administering insulin to his or herself, for example, via an insulin pen. A closed loop system (also referred to as an artificial pancreas) is a system that monitors blood glucose levels automatically and provides insulin to a PWD. For example, a CGM monitors blood glucose levels of a PWD and an insulin pump (communicative coupled to the CGM) delivers insulin to the PWD. Additionally, in some embodiments, insulin delivery system includes a mobile device (e.g., PWD device 130).

Therapy management system 1100B includes PWD engine 126, HCP engine 128, insights/recommendation engine 1160 and therapy management engine 1140. In various embodiments, insights/recommendation engine 1160 includes HCP engine 128 and recommendation engine 124. Insights/recommendation engine 1160, in various embodiments, receives PWD data 106 insulin delivery system 1150 and/or therapy management engine 1140. For example, therapy management engine 1140 receives PWD data 106 from insulin delivery system 1150. Upon receiving PWD data 106 from insulin delivery system 1150, therapy management engine 1140 transmits PWD data 106 to insights/recommendation engine 1160.

Insights/recommendation engine 1160 generates insights and/or recommendations, as described herein, based, at least in part, on data from insulin delivery system 1150 and/or therapy management engine 1140. In various embodiments, insights may be generated without knowing the amount of insulin delivered. For example, the insights may be generated relying on system recommended dosing (i.e. meal & correction vs just meal). In such an example, insights/recommendation engine 1160 generates insights/recommendations based on rules to distinguish which insulin and direction to change (e.g., RA meal increase, RA correction decrease, and LA decrease).

Embodiments of therapy management system depicted in FIGS. 11A-B, and methods of configuring and operating a therapy management system, may be performed, in whole or in part, in cloud computing, client-server, or other networked environment, or any combination thereof. The components of such a system may be located in a singular “cloud” or network, or spread among many clouds or networks. End-user knowledge of a physical location and/or configuration of components of a system are not required.

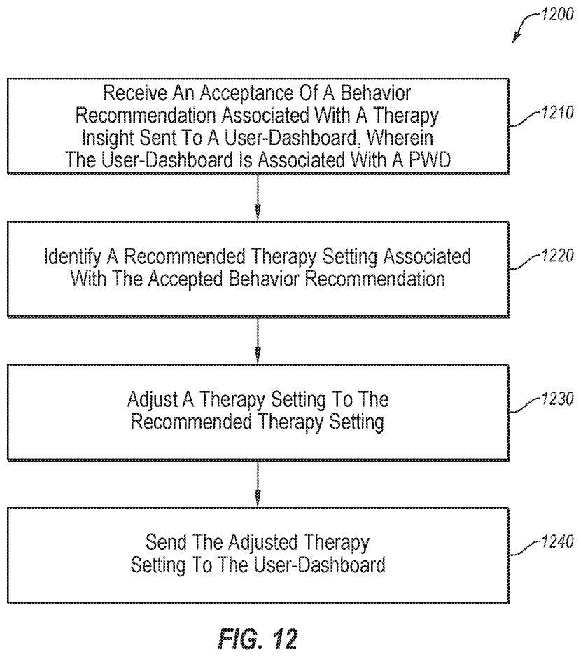

FIG. 12 illustrates a method 1200 for managing therapy settings for a PWD. In various embodiments, method 1200 is implemented, at least based in part, by therapy management system 1000 and/or therapy management system 1100. In various embodiments, method 1200 is implemented, at least based in part, by CDS system 100A.

At operation 1210, an acceptance of a behavior recommendation associated with a therapy insight sent to a user-dashboard is received. For example, one or more behavior recommendations are presented at a PWD dashboard. A user selects one or more of the behavior recommendations that are presented at a PWD dashboard.

At operation 1220, a recommended therapy setting associated with the accepted behavior recommendation is identified. Upon selection of a recommendation, in one embodiment, therapy management engine 1140 identifies a recommended therapy setting associated with the accepted behavior recommendation. For example, referring to FIGS. 5 and 11, therapy management engine 1140 identifies a recommended setting change 531 associated with one or more of selected recommendations 420 (selected by a PWD).

At operation 1230, a therapy setting to the recommended therapy setting is adjusted. In one embodiment, referring once again to FIGS. 5 and 11, therapy management engine 1140 adjusts a first therapy setting, such as a large meal insulin dose of 5 units (e.g. a scheduled dose) to a second, different therapy setting of a large meal insulin dose of 4 units.

In another embodiment, therapy management engine 1140 sends a recommended therapy setting to mobile application 1125 that displays the recommended therapy setting. Upon selection of the recommended therapy setting by a PWD, mobile application 1125 adjusts a therapy setting to the recommended therapy setting.

At operation 1240, the adjusted therapy setting is sent to the user-dashboard. For example, upon a therapy setting adjusted to a recommended therapy setting (e.g., a large meal insulin dose of 5 units is adjusted to a dose of 4 units), the adjusted therapy setting is sent to PWD dashboard 132.

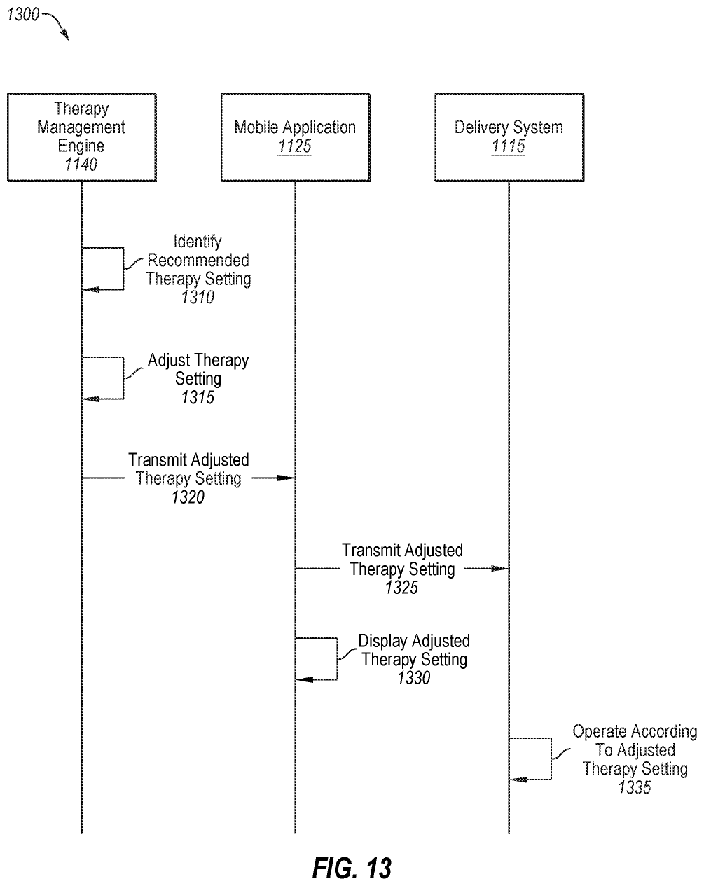

FIG. 13 illustrates a method 1300 for managing therapy settings for a PWD.

At operation 1310, therapy management engine 1140 identifies a recommended therapy setting. For example, a PWD selects a behavioral recommendation provided by a HCP. Therapy management engine 1140 then identifies a recommended therapy setting (e.g., reducing correction insulin by 20%) associated with the behavioral recommendation.

At operation 1315, upon identifying the recommended therapy setting, therapy management engine 1140 adjusts the therapy setting. For example, therapy management engine 1140 adjusts a current therapy setting of correction insulin (e.g., 10 units) to an adjusted therapy setting (e.g., 8 units).

At operation 1315, therapy management engine 1140 sends the adjusted therapy setting to mobile application 1125.

At operation 1325, mobile application 1125 transmits the adjusted therapy setting to delivery system 1115 for implementation of the adjusted therapy setting by the delivery system (e.g., providing 8 units of correction insulin to a PWD). In one embodiment, mobile application 1125 sends the adjusted therapy setting to delivery system upon acceptance of the adjusted therapy setting by a PWD. In another embodiment, mobile application 1125 sends the adjusted therapy setting to delivery system without prompting a PWD for acceptance of the adjusted therapy setting.

At operation 1330, mobile application 1125 indicates to a PWD that a therapy setting has been adjusted.

At operation 1335, delivery system 1115 (e.g., insulin pump) operates according to the adjusted therapy setting. For example, delivery system 1115 injects a PWD with insulin according to the adjusted therapy setting (e.g., 8 units of correction insulin).

The embodiments described herein may include the use of a special-purpose or general-purpose computer including various computer hardware or software modules, as discussed in greater detail below.

Embodiments described herein may be implemented using computer-readable media for carrying or having computer-executable instructions or data structures stored thereon. Such computer-readable media may be any available media that may be accessed by a general-purpose or special-purpose computer. Special-purpose computer is intended to be interpreted broadly and encompasses embedded systems, microcontrollers, application specific integrated circuits, digital signal processors, and general-purpose computers programmed for specific purposes. Segments (e.g., code segment or data segment) may refer to a portion (e.g., address) of memory, virtual memory, or an object file.

By way of example, and not limitation, such computer-readable media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid-state memory devices), or any other storage medium which may be used to carry or store desired program code in the form of computer-executable instructions or data structures and which may be accessed by a general-purpose or special-purpose computer. Combinations of the above may be included within the scope of computer-readable media.

Computer-executable instructions comprise, for example, instructions and data which cause a general-purpose computer, special-purpose computer, or special-purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Any ranges expressed herein (including in the claims) are considered to be given their broadest possible interpretation. For example, unless explicitly mentioned otherwise, ranges are to include their endpoints (e.g., a range of “between X and Y” would include X and Y). Additionally, ranges described using the terms “approximately” or “about” are to be understood to be given their broadest meaning consistent with the understanding of those skilled in the art. Additionally, the terms “approximately” or “substantially” include anything within 10%, or 5%, or within manufacturing or typical tolerances.

The features of the various embodiments described herein are not mutually exclusive and can exist in various combinations and permutations, even if such combinations or permutations are not expressly described herein, without departing from the scope of the disclosure. In fact, variations, modifications, and other implementations of what is described herein will occur to one of ordinary skill in the art without departing from the scope of the disclosure. As such, the invention is not to be defined only by the preceding illustrative description, but only by the claims which follow, and legal equivalents thereof.

1. A computer-implemented method for automated provisioning of clinical advice to a provider caring for a patient with diabetes (PWD), comprising:
detecting a clinically relevant pattern in insulin therapy data of a patient under the care of a provider;
identifying a predefined behavior of a PWD responsive to the detected clinically relevant pattern, wherein the clinically relevant detected pattern is indicative of desirable behaviors and/or undesirable behaviors related in perspective of insulin-based management of diabetes; and
selecting a therapy insight associated with the identified predefined behavior, wherein the therapy insight comprises clinical advice associated with the PWD that includes a behavior recommendation; and
responsive to the selecting the therapy insight, automatically sending, to a provider-dashboard associated with the provider caring for the PWD, the selected therapy insight.

2. The computer-implemented method of claim 1, wherein sending the selected therapy insight comprises:
sending one or more of a behavior recommendation to adjust the undesirable behaviors and a behavior recommendation to continue the desirable behaviors.

3. The computer-implemented method of claim 1, wherein sending the selected insight comprises:
sending one or more of a long acting dosing recommendation and a rapid acting dosing recommendation.

4. The computer-implemented method of claim 1, wherein identifying a predefined behavior of a PWD further comprises:
identifying whether one or more of a scheduled dose of insulin was administered to the PWD and an incorrect dose of insulin was administered to the PWD.

5. The computer-implemented method of claim 1, further comprising:
correlating therapy insights to associated health risks; and
prioritizing the therapy insights based on a severity of the health risks.

6. The computer-implemented method of claim 1, further comprising:
prioritizing therapy insights based on frequency of occurrences of the predefined behavior during a predetermined time frame.

7. The computer-implemented method of claim 1, further comprising:
responsive to determining that the insulin therapy data does not correspond to a clinically relevant pattern, forgo identifying the predefined behavior.

8. The computer-implemented method of claim 1, wherein the insulin therapy data is selected from a group consisting of: glucose data, therapy settings and insulin dosing data.

9. The computer-implemented method of claim 1, wherein the insulin therapy data is glucose measurements continuously received, wherein at least some of the glucose measurements includes date, time and glucose value.

10. The computer-implemented method of claim 1, wherein the insulin therapy data is dosing information that includes date, time and amount of a dose of insulin.

11. The computer-implemented method of claim 1, further comprising:
receiving selection of one or more behavior recommendations associated with the therapy insight sent to the provider-dashboard.

12. The computer-implemented method of claim 1, further comprising:
receiving an indication of cancelation of one or more behavior recommendations associated with the therapy insight sent to the provider-dashboard.

13. The computer-implemented method of claim 1, further comprising:
receiving adjustments of the therapy insight sent to the provider-dashboard.

14. A system for automated provisioning of clinical advice to a provider caring for a patient with diabetes (PWD) comprising:
an insights engine configured to:
detect a clinically relevant pattern in insulin therapy data of a patient under the care of a provider;
identify a predefined behavior of a PWD responsive to the detected clinically relevant pattern, wherein the clinically relevant detected pattern is indicative of desirable behaviors and/or undesirable behaviors related in perspective of insulin-based management of diabetes; and
select a therapy insight associated with the identified predefined behavior, wherein the therapy insight comprises clinical advice associated with the PWD that includes a behavior recommendation; and
a health care provider (HCP) engine configured to automatically send, to a provider-dashboard associated with the provider caring for the PWD, the selected therapy insight.

15. The system of claim 14, wherein the HCP engine is further configured to send one or more of a behavior recommendation to adjust the undesirable behaviors and a behavior recommendation to continue the desirable behaviors.

16. The system of claim 14, wherein the HCP engine is further configured to send one or more of a behavior recommendation to adjust the undesirable behaviors and a behavior recommendation to continue the desirable behaviors.

17. The system of claim 14, wherein the HCP engine is further configured to receive selection of one or more behavior recommendations associated with the therapy insight sent to the provider-dashboard.

18. The system of claim 14, wherein the HCP engine is further configured to receive an indication of cancelation of one or more behavior recommendations associated with the therapy insight sent to the provider-dashboard.

19. The system of claim 14, wherein the HCP engine is further configured to receive adjustments of the therapy insight sent to the provider-dashboard.

20. The system of claim 14, wherein the insights engine is further configured to identify whether one or more of a scheduled dose of insulin was administered to the PWD and an incorrect dose of insulin was administered to the PWD.

21. The system of claim 14, wherein the insights engine is further configured to:
correlate therapy insights to associated health risks; and
prioritize the therapy insights based on a severity of the health risks.

22. The system of claim 14, wherein the insights engine is further configured to prioritize therapy insights based on frequency of occurrences of the predefined behavior during a predetermined time frame.

23. The system of claim 14, wherein the insights engine is further configured to responsive to determining that the insulin therapy data does not correspond to a clinically relevant pattern, forgo identifying the predefined behavior.

24. The system of claim 14, wherein the insulin therapy data is selected from a group consisting of: glucose data, therapy settings and insulin dosing data.

25. The system of claim 14, wherein the insulin therapy data is glucose measurements continuously received, wherein at least some of the glucose measurements includes date, time and glucose value.

26. The system of claim 14, wherein the insulin therapy data is dosing information that includes date, time and amount of a dose of insulin.

27. A computer-implemented method for managing insulin therapy settings for a patient with diabetes (PWD), comprising:
receiving an acceptance of a behavior recommendation associated with a therapy insight sent to a user-dashboard, wherein the user-dashboard is associated with a PWD;
identifying a recommended insulin therapy setting associated with the accepted behavior recommendation;
adjusting an insulin therapy setting to the recommended insulin therapy setting; and
sending the adjusted insulin therapy setting to the user-dashboard.

28. The computer-implemented method of claim 27, wherein the sending the adjusted insulin therapy setting to the user-dashboard is to automatically adjust the insulin therapy setting to the recommended insulin therapy setting.

29. The computer-implemented method of claim 27, wherein the sending the adjusted insulin therapy setting to the user-dashboard is to be displayed on the user-dashboard.

30. The computer-implemented method of claim 27, further comprising:
operating an insulin delivery system with the adjusted insulin therapy setting.

31. A computer-implemented method for managing insulin therapy settings for a patient with diabetes (PWD), comprising:
receiving an acceptance to adjust an insulin therapy setting displayed on a user-dashboard associated with a PWD, wherein the insulin therapy setting is associated with a behavior recommendation for the PWD;
automatically adjusting the insulin therapy setting corresponding to the accepted adjusted insulin therapy setting; and
sending the adjusted insulin therapy setting to the user-dashboard.

32. The computer-implemented method of claim 31, further comprising:
operating an insulin delivery system with the adjusted insulin therapy setting.

33. A computer-implemented method for automated provisioning of clinical advice to a provider caring for a patient with diabetes (PWD), comprising:
identifying, by an insights engine associated with a first computing system, a predefined behavior of a PWD responsive to a detected clinically relevant pattern in insulin therapy data;
selecting, by the insights engine, a therapy insight associated with the identified predefined behavior; and
responsive to the selecting the therapy insight, automatically sending over a network, to a health care provider engine associated with a second computing system separate and distinct from the first computing system, the selected therapy insight, wherein the health care provider engine is configured to automatically send the therapy insight to a provider-dashboard associated with a provider caring for the PWD.

34. The computer-implemented method of claim 33, further comprising:
detecting the clinically relevant pattern in insulin therapy data of a patient under the care of a provider.

35. The computer-implemented method of claim 33, further comprising:
receiving a selected therapy insight from the health care provider engine; and
transmitting the selected therapy insight to a PWD dashboard.
